(19) United States
(12) Patent Application Publication
(10) Pub. No.: US 2006/0247286 A1
(43) Pub. Date: Nov. 2, 2006

(54) Oxazolidinone antibiotics and derivatives thereof

(76) Inventors: Milton Hammond, Branchburg, NJ (US); Yasumichi Fukuda, Tochigi (JP)

Correspondence Address: MCDERMOTT WILL & EMERY LLP, 600 13TH STREET, N.W., WASHINGTON, DC, 20005-3096, US

(21) Appl. No.: 10/559,845

(22) PCT Filed: Jun. 29, 2004
(86) PCT No.: PCT/US04/20736

§ 371 Date: May 5, 2006

Related U.S. Application Data
(60) Provisional application No. 60/483,905, filed on Jul. 2, 2003.
(60) Provisional application No. 60/546,947, filed on Feb. 24, 2004.
(60) Provisional application No. 60/553,963, filed on Mar. 18, 2004.

(51) Int. Cl. A61K 31/422; C07D 413/02

(52) U.S. Cl. 514/376; 548/229

(57) ABSTRACT

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.
The compounds are represented by structural formula I: 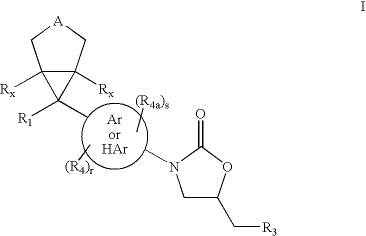 its enantiomer, diastereomer, or pharmaceutically acceptable salt or ester thereof.

### CROSS REFERENCE TO RELATED APPLICATIONS

This application claims the benefit of U.S. Provisional Application No. 60/483,905, filed Jul. 2, 2003, entitled OXAZOLIDINONE ANTIBIOTICS AND DERIVATIVES THEREOF; U.S. Provisional Application No. 60/546,947, filed Feb. 2, 2004, entitled OXAZOLIDINONE ANTIBIOTICS AND DERIVATIVES THEREOF; and U.S. Provisional Application No. 60/553,963, filed Mar. 18, 2004, entitled OXAZOLIDINONE ANTIBIOTICS AND DERIVATIVES THEREOF, which are hereby incorporated herein by reference in their entirety.

### BACKGROUND OF THE INVENTION

Oxazolidinones represent the first new class of antibacterials to be developed since the quinolones. The oxazolidinones are synthetic antibacterial compounds that are orally or intravenously active against problematic multidrug resistant Gram positive organisms and are not cross-resistant with other antibiotics. See Riedl et al, Recent Developments with Oxazolidinone Antibiotics, Exp. Opin. Ther. Patents (1999) 9(5), Ford et al., Oxazolidinones: New Antibacterial Agents, Trends in Microbiology 196 Vol.5, No. 5, May 1997 and WO 96/35691. See also WO 03/063862, WO 01/81350, WO 01/94342, WO 03/072553, EP 0352781 and U.S. Pat. Nos. 5,565,571 and 4,053,593.

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.

### SUMMARY OF THE INVENTION

The present invention relates to compounds of formula I: 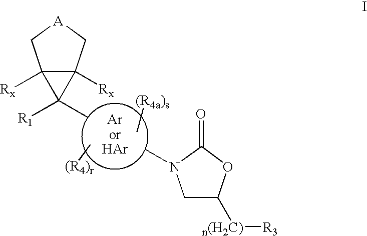
its enantiomer, diastereomer, or pharmaceutically acceptable salt, hydrate or prodrug thereof wherein:
R₁ represents
   i) hydrogen,
   ii) NR₅R₆,
   iii) CR₇R₈R₉, C(R)₂OR₁₄, CH₂NHR₁₄,
   iv) C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄,
   v) (C₁₋₆alkyl)CN, CN, CH═C(R)₂, (CH₂)ₚOH, C(═O)CHR₁₃, C(═NR₁₃)R₁₃, NR₁₀C(═X₁)R₁₃; or
   vi) C₅₋₁₀ heterocycle optionally substituted with 1-3 groups of R₇, which may be attached through either a carbon or a heteroatom;

A represents NR, O, or S(O)ₚ; 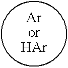
represents aryl or heteroaryl, heterocycle, heterocyclyl or heterocyclic, provided that in the case of a heteroaryl, heterocycle, heterocyclyl or heterocyclic, the cyclopropyl is not attached to a nitrogen atom on the ring;
Rₓ represents hydrogen or C₁₋₆ alkyl;
R₃ represent
   i) NR₁₃(C═X₂)R₁₂,
   ii) NR₁₃(C═X₁)R₁₂,
   iii) NR₁₃SO₂R₁₄,
   iv) N(R₁₃)heteroaryl,
   v) NR₁₃(CHR₁₃)₀₋₄aryl,
   vi) NR₁₃(CHR₁₃)₀₋₄heteroaryl,
   vii) S(CHR₁₃)₀₋₄aryl,
   viii) S(CHR₁₃)₀₋₄heteroaryl,
   ix) O(CHR₁₃)₀₋₄aryl,
   x) O(CHR₁₃)₀₋₄heteroaryl,
   xi) NOH(C═X₁)R₂,
   xii) —OC═N(OCOaryl)C₁₋₆ alkyl
   xiii) —OC═N(OH)C₁₋₆ alkyl
   xiv) C₅₋₁₀heteroaryl which may be attached through either a carbon or a heteroatom; said aryl and heteroaryl optionally substituted with 1-3 groups of R₇,
R₄, and R₄ₐ, independently represent
   i) hydrogen,
   ii) halogen,
   iii) C₁₋₆ alkoxy, or
   iv) C₁₋₆ alkyl
r and s independently are 1-3, with the provision that when (R₄ₐ)ₛ and (R₄)ᵣ are attached to an Ar or HAr ring the sum of r and s is less than or equal to 4;
R₅ and R₆ independently represent
   i) hydrogen,
   ii) C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ allylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;
   iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
   iv) C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl;
   v) arylsulfonyl optionally substituted with 1-3 of halogen, C₁₋₆ alkoxy, OH or C₁₋₆ alkyl;
   vi) C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl;
   vii) aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl
   viii) five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
   ix) C₃₋₆ cycloalkylcarbonyl optionally substituted with 1-3 groups of halogen, OH, C₁₋₆ alkoxy or CN;
   x) benzoyl optionally substituted with 1-3 groups of halogen, OH, C₁₋₆ alkoxy, C₁₋₆ alkyl, CF₃, C₁₋₆ alkanoyl, amino or C₁₋₆ acylamino;
   xi) pyrrolylcarbonyl optionally substituted with 1-3 of C₁₋₆ alkyl;
   xii) C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or
R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
   i) hydrogen, halogen, CN, CO₂R, CON(R)₂, CHO, CH₂NHAc, C(═NOR), OH, C₁₋₆ alkoxy, C₁₋₆ alkyl, alkenyl, hydroxy C₁₋₆ alkyl, (CH₂)₁₋₃NHC(O)C₁₋₆ alkyl, (CH₂)₁₋₃ N(C₁₋₆ alkyl)₂
   ii) (CH₂)ₙamino, (CH₂)ₙC₁₋₆ alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
   i) H, CN,
   ii) C₁₋₆ alkyl optionally substituted with 1-3 halogen, CN, OH, C₁₋₆ alkoxy, C₁₋₆ acyloxy, or amino,
   iii) phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
X₂ represents O, S, NH or NSO₂R₁₄;
R₁₀ represents hydrogen, C₁₋₆ allyl or CO₂R₁₅;
R₁₂ represents hydrogen, C₁₋₆ alkyl, NH₂, OR, CHF₂, CHCl₂, CR₂Cl, (CH₂)ₙSR, (CH₂)ₙCN, (CH₂)ₙSO₂R, (CH₂)ₙS(O)R, C₁₋₆ alkylamino, C₅₋₁₀ heteroaryl or C₁₋₆ dialkylamino, where said alkyl may be substituted with 1-3 groups of halo, CN, OH or C₁₋₆ alkoxy, said heteroaryl optionally substituted with 1-3 groups of R₇;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, —OCOaryl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen, (CH₂)ₚCN, C₁₋₆ alkyl, CO₂C₁₋₆ alkyl, COCH₂OH, COCH₂OCOC₁₋₆ alkyl, SO₂C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
p represents 0-2 and
m, n, and q represents 0-1.

Another aspect of the invention is concerned with the use of the novel antibiotic compositions in the treatment of bacterial infections.

### DETAILED DESCRIPTION OF THE INVENTION

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994, in particular pages 1119-1190).

When any variable (e.g. aryl, heterocycle, R₅, R₆ etc.) occurs more than once, its definition on each occurrence is independent at every other occurrence. Also combinations of substituents/or variables are permissible only if such combinations result in stable compounds.

The term “alkyl” refers to a monovalent alkane (hydrocarbon) derived radical containing from 1 to 15 carbon atoms unless otherwise defined. It may be straight or branched. Preferred alkyl groups include lower alkyls which have from 1 to 6 carbon atoms such as methyl, ethyl, propyl, isopropyl, butyl and t-butyl. When substituted, alkyl groups may be substituted with up to 3 substituent groups, selected from the groups as herein defined, at any available point of attachment. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused Preferred cycloalkyl groups are cyclopropyl, cyclobutyl, cyclopentyl and cyclohexyl. When substituted, cycloalkyl groups may be substituted with up to 3 substituents which are defined herein by the definition of alkyl.

Alkanoyl refers to a group derived from an aliphatic carboxylic acid of 2 to 4 carbon atoms. Examples are acetyl, propionyl, butyryl and the like.

The term “alkoxy” refers to those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like. 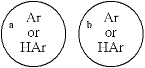
refers to aryl or heteroaryl, heterocycle, Het, heterocyclyl or heterocyclic as described immediately below.

Aryl refers to any stable monocyclic or bicyclic carbon ring of up to 7 atoms in each ring, wherein at least one ring is aromatic. Examples of such aryl elements include phenyl, napthyl, tetrahydronaphthyl, indanyl, indanonyl, biphenyl, tetralilnyl, tetralonyl, fluorenonyl, phenanthryl, anthryl, acenaphthyl, and the like substituted phenyl and the like. Aryl groups may likewise be substituted as defined. Preferred substituted aryls include phenyl and naphthyl.

The term heterocycle, heteroaryl, Het, heterocyclyl or heterocyclic, as used herein except where noted, represents a stable 5- to 7-membered mono- or bicyclic or stable 8- to 11-membered bicyclic heterocyclic ring system, any ring of which may be saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized (in which case it is properly balanced by a counterion), and including any bicyclic group in which any of the above-defined heterocyclic rings is fused to a benzene ring. The heterocyclic ring may be attached at any heteroatom or carbon atom, which results in the creation of a stable structure. The term heterocycle or heterocyclic includes heteroaryl moieties. “Heterocycle” or “heterocyclyl” therefore includes the above mentioned heteroaryls, as well as dihydro and tetrahydro analogs thereof. The heterocycle, heteroaryl, Het or heterocyclic may be substituted with 1-3 groups of R₇. Examples of such heterocyclic elements include, but are not limited to the following: piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyrimidonyl, pyridinonyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thiophenyl, imidazopyridinyl, triazolyl, tetrazolyl, triazinyl, thienyl, benzothienyl, thiamorpholinyl sulfoxide, thiamorpholinyl sulfone, naphthpyridinyl, dihydropyrinidinyl, dihydropyrrolyl, dihydroquinolinyl, dihydrotetrazolyl, dihydrotriazolyl, dihydrothienyl, dihydrooxazolyl, dihydrobenzothiophenyl, dihydrofuranyl, benzothiazolyl, benzothienyl, benzoimidazolyl, benzopyranyl, benzothiofuranyl, carbolinyl, chromanyl, cinnolinyl, benzopyrazolyl, benzodioxolyl and oxadiazolyl. Additional examples of heteroaryls are illustrated by formulas a, b, c and d: 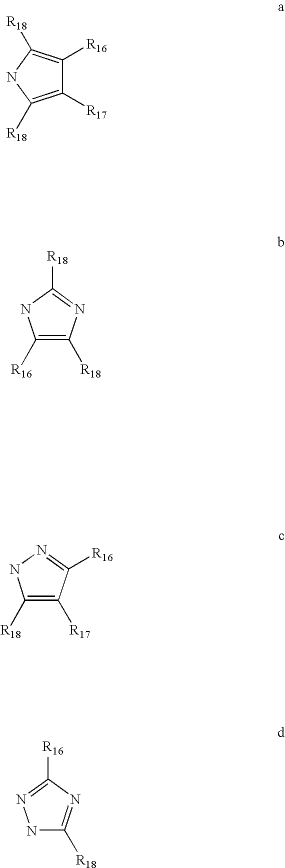
wherein R₁₆ and R₁₇ are independently selected from hydrogen, halogen, C₁₋₆ alkyl, C₂₋₄ alkanoyl, C₁₋₆ alkoxy; and R₁₈ represents hydrogen, C₁₋₆ alkyl, C₂₋₄ alkanoyl, C₁₋₆ alkoxycarbonyl and carbamoyl.

The term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl.

The terms “quaternary nitrogen” and “positive charge” refer to tetravalent, positively charged nitrogen atoms (balanced as needed by a counterion known in the art) including, e.g., the positively charged nitrogen in a tetraalkylammonium group (e. g. tetramethylammonium), heteroarylium, (e.g., N-methyl-pyridinium), basic nitrogens which are protonated at physiological pH, and the like. Cationic groups thus encompass positively charged nitrogen-containing groups, as well as basic nitrogens which are protonated at physiologic pH.

The term “heteroatom” means O, S or N, selected on an independent basis.

The term “prodrug” refers to compounds which are drug precursors which, following administration and absorption, release the drug in vivo via some metabolic process. Exemplary prodrugs include acyl amides of the amino compounds of this inventon such as amides of alkanoic(C₁₋₆)acids, amides of aryl acids (e.g., benzoic acid) and alkane(C₁₋₆)dioic acids.

Halogen and “halo” refer to bromine, chlorine, fluorine and iodine.

When a group is termed “substituted”, unless otherwise indicated, this means that the group contains from 1 to 3 substituents thereon.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

Examples of suitable hydroxyl and amino protecting groups are: trimethylsilyl, triethylsilyl, o-nitrobenzyloxycarbonyl, p-nitrobenzyloxycarbonyl, t-butyldiphenylsilyl, t-butyldimnethylsilyl, benzyloxycarbonyl, t-butyloxycarbonyl, 2,2,2-trichloroethyloxycarbonyl, allyloxycarbonyl and the like. Examples of suitable carboxyl protecting groups are benzhydryl, o-nitrobenzyl, p-nitrobenzyl, 2-naphthylmethyl, allyl, 2-chloroallyl, benzyl, 2,2,2-trichloroethyl, trimethylsilyl, t-butyldimethylsilyl, t-butldiphenylsilyl, 2-(trimethylsilyl)ethyl, phenacyl, p-methoxybenzyl, acetonyl, p-methoxyphenyl, 4-pyridylmethyl, t-butyl and the like.

The cyclopropyl containing oxazolidinone compounds of the present invention are useful per se and in their pharmaceutically acceptable salt and ester forms for the treatment of bacterial infections in animal and human subjects. The term “pharmaceutically acceptable ester, salt or hydrate,” refers to those salts, esters and hydrated forms of the compounds of the present invention which would be apparent to the pharmaceutical chemist i.e., those which are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug. Conveniently, pharmaceutical compositions may be prepared from the active ingredients in combination with pharmaceutically acceptable carriers. Thus, the present invention is also concerned with pharmaceutical compositions and methods of treating bacterial infections utilizing as an active ingredient the novel cyclopropyl containing oxazolidinone compounds.

The pharmaceutically acceptable salts referred to above also include acid addition salts. Thus, when the Formula I compounds are basic, salts may be prepared from pharmaceutically acceptable non-toxic acids, including inorganic or organic acids. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, funarate, glucoheptanoate, glycerophosphate, hemisulfate, heptanoate, hexanoate, hydrochloride, hydrobromide, hydroiodide, 2-hydroxyethanesulfonate, isethionic, lactate, maleate, mandelic, malic, maleic, methanesulfonate, mucic, 2-naphthalenesulfonate, nicotinate, nitric oxalate, pamoate, pectinate, persulfate, 3-phenylpropionate, picrate, pivalate, propionate, phosphate, pantothenic, paamoic, sulfate, succinate, tartrate, thiocyanate, tosylate and undecanoate.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium zinc and the like. Particularly preferred are the ammonium, calcium, magnesium, potassium and sodium salts. Salts derived from pharmaceutically acceptable inorganic non-toxic bases include salts of primary, secondary and teritary amines, substituted amines including naturally occurring substituted amines, cyclic amines and basic ion exchange resins, such as arginine, betaine caffeine, choline, N,N¹-dibenzylethylenediamime, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The pharmaceutically acceptable esters are such as would be readily apparent to a medicinal chemist, and include those which are hydrolyzed under physiological conditions, such as “biolabile esters”, pivaloyloxymethyl, acetoxymethyl, phthalidyl, indanyl and methoxymethyl, and others.

Biolabile esters are biologically hydrolizable, and may be suitable for oral administration, due to good absorption through the stomach or intenstinal mucosa, resistance to gastric acid degradation and other factors. Examples of biolabile esters include compounds.

An embodiment of this invention is realized when R₁ represents H, NR₅R₆, CN, OH, C(R)₂OR₁₄, NHC(═X1)N(R₁₃)₂, C(═NOH)N(R₁₃)₂, NR₁₀C(═X₁)R₁₃ or CR₇R₈R₉ and all other variables are as described herein.

Another embodiment of this invention is realized when 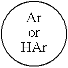
is phenyl, pyridine, pyrimidine, or piperidine and all other variables are as described herein.

Another embodiment of this invention is realized when one of R₁ is NR₁₀C(═X₁)R₁₃ and and all other variables are as described herein.

Another embodiment of this invention is realized when one of R₁ is CN and all other variables are as described herein.

Another embodiment of this invention is realized when one of R₁ NR₅R₆ and all other variables are as described herein.

Another embodiment of this invention is realized when R₃ is NR(C═X₁)R₁₂, C₅₋₁₀ heteroaryl, NH(CH₂)₀₋₄aryl, NH(CH₂)₀₋₄heteroaryl, said aryl and heteroaryl optionally substituted with 1-3 groups of Ra and all other variables are as described herein.

Another embodiment of this invention is realized when R₃ is a C₅₋₁₀ heteroaryl represented by 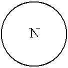
which represents an optionally substituted aromatic heterocyclic group containing 1 to 4 nitrogen atoms and at least one double bond, and which is connected through a bond on any nitrogen. Exemplary groups are 1,2,3-triazole, 1,2,4-triazole, 1,2,5-triazole, tetrazole, pyrazole, and imidazole, any of which may contain 1 to 3 substitutents selected from R₇.

Still another embodiment of this invention is realized when R₅ and R₆ independently are:
i) H,
ii) C₁₋₆ alkyl optionally substituted with 1-3 groups of halogen, CN, OH, C₁₋₆ alkoxy, amino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethyenyloxy, or ethynyl, said phenyl and pyridine optionally substituted with 1-3 halogen, CN, OH, CF₃, C₁₋₆ alkyl or C₁₋₆ alkoxy;
iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl; or
iv) benzoyl optionally substituted with 1-3 groups of halogen, OH, C₁₋₆ alkoxy, C₁₋₆ alkyl, CF3, C₁₋₆ alkanoyl, amino or C₁₋₆ acylamino and all other variables are as described herein.

Yet another embodiment of this invention is realized when X₁ represents O and all other variables are as described herein.

A preferred embodiment of this invention is realized when the structural formula is II: 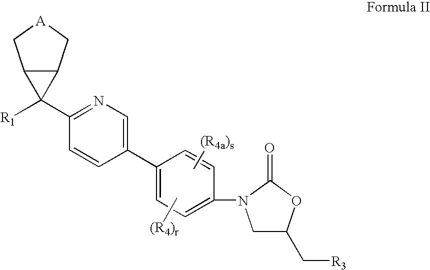
wherein R₁, R₄, R₄ₐ, Y and R₃ are as described herein.

Preferred compounds of this invention are:
N-[5(S)-3-[4[(1α,5α,6β)-6-cyanobicyclo[3.1.0]hexan-6-yl)]phenyl]-2-oxooxazolidin-5-ylmethyl]acetamide,
1-[5(R)-3-[4-[(1α,5α,6β)-(6-cyanobicyclo[3.1.0]hexan-6-yl)]phenyl]-2-oxooxazolidin-5-ylmethyl]-1,2,3-triazole,
N-[5(S)-3-[4-[(1α,5α,6β)-3-t-butoxycarbonyl-6-cyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-azabicyclo[3.1.0]hexan-6-yl)]phenyl]-2-oxooxazolidin-5-ylmethyl]acetamide,
1-[5(R)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]-1,2,3-triazole,
1-[5(R)-3-[4-[(1α,5α,6β)-6-cyano-3-azabicyclo[3.1.0]hexan-6-yl)]phenyl]-2-oxooxazolidin-5-ylmethyl]-1,2,3-triazole,
N-[5(S)-3-[4-[(1α,5α,6β)-(3-acetoxyacetyl-6-cyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-hydroxyacetyl-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-methanesulfonyl-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-methyl-3-azabicyclo[3.1.0]hexan-6-yl)]phenyl]-2-oxooxazolidin-5-ylmethyl]acetamide,
N-[5(S)-3-[4-[(1α,5α,6β)-(3,6-dicyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-cyanomethyl-3-azabicyclo[3.1.0]hexan-yl)]phenyl]-2-oxooxazolidin-5-ylmethyl]acetamide,
5(R)-3-[4-[(1α,5α,6β)-(3-t-butoxycarbonyl-6-cyano-3-azabicyclo[3.1.0]hexan-6-yl)]phenyl]-5-[(isoxazol-3-yl)oxy]methyloxazolidin-2-one,
5(R)-3-[4-[(1α,5α,6β)-(6-cyano-3-azabicyclo[3.1.0]hexan-yl)]phenyl]-5-[(isoxazol-3-yl)oxy]methyloxazolidin-2-one,
5(R)-3-[4-[(1α,5α,6β)-(3-t-butoxycarbonyl-6-cyano-3-azabicyclo[3.1.0]hexan-6-yl)]phenyl]-5-[N-(t-butoxycarbonyl)-N-(isoxazol-3-yl)]amiomethyloxazolidin-2-one,
5(R)-3-[4-[(1α,5α,6β)-(6-cyano-3-azabicyclo[3.1.0]hexan-yl)]phenyl]-5-[N-(isoxazol-3-yl)]aminomethyloxazolidin-2-one,
N-[5(S)-3-[4-[(1α,5α,6β)-[6-cyano-3-5-cyanopyridin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(pyridin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[3-acetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(pyrimidin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(4-pyridylmethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyano-1-iminoethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-methoxycarbonyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyano-S-methylthioiminomethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyanocarboxamidyl)-3-azabicyclo[3.1.0]hexan-6-yl]]phenyl]-2-oxooxazolidin-5-ylmethyl]acetamide,
N-[5(S)-3-[4-[(1α,5α,6β)-[3-(N,N′-t-butoxycarbonylcarboxamidyl)-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-carboxamidyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-N-t-Butoxycarbonylamino)acetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-aminoacetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-methanesulfonylacetyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-dibenzylphosphoryloxy)acetyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(phosphoryloxy)acetyl-3-azabicyclo[3.1.0]hexan-6-yl]]phenyl]-2-oxooxazolidin-5-ylmethyl]acetamide,
   or their enantiomer, diastereomer, or pharmaceutically acceptable salt, hydrate or prodrug thereof wherein.

Suitable subjects for the administration of the formulation of the present invention include mammals, primates, man, and other animals. In vitro antibacterial activity is predictive of in vivo activity when the compositions are administered to a mammal infected with a susceptible bacterial organism.

Using standard susceptibility tests, the compositions of the invention are determined to be active against MRSA and enterococcal infections.

The compounds of the invention are formulated in pharmaceutical compositions by combining the compounds with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, a preferred route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

The novel antibiotic compositions of this invention for human delivery per unit dosage, whether liquid or solid, comprise from about 0.01% to as high as about 99% of the cyclopropyl containing oxazolidinone compounds discussed herein, the preferred range being from about 10-60% and from about 1% to about 99.99% of one or more of other antibiotics such as those discussed herein, preferably from about 40% to about 90%. The composition will generally contain from about 125 mg to about 3.0 g of the cyclopropyl containing oxazolidinone compounds discussed herein; however, in general, it is preferable to employ dosage amounts in the range of from about 250 mg to 1000 mg and from about 200mg to about 5 g of the other antibiotics discussed herein; preferably from about 250 mg to about 1000 mg. In parenteral administration, the unit dosage will typically include the pure compound in sterile water solution or in the form of a soluble powder intended for solution, which can be adjusted to neutral pH and isotonic.

The invention described herein also includes a method of treating a bacterial infection in a mammal in need of such treatment comprising administering to said mammal the claimed composition in an amount effective to treat said infection.

Oxazolidinones have been known at times to cause side effects such as sideroblastic anemia, peripheral sensory neuropathy, optic neuropathy, seizures, thrombocytopenia, cheilosis, seborrheic dermatitis, hypo-regenerative anemia, megaloblastic anemia or normocytic anemia The compounds of the invention may be combined with an effective amount of one or more vitamins to prevent or reduce the occurrence of oxazolidinone-associated side effects in patients. The vitamins that can be combined are vitamin B2, vitamin B6, vitaimin B12 and folic acid. The vitamins may be administered with the oxazolidinones as separate compositions or the vitamins and oxazolidinones may be present in the same composition.

Thus another aspect of this invention is a method of treating or preventing an oxazolidinone-associated side effect by administering an effective amount of the oxazolidinone of structural formula I and an effective amount of one or more of vitamin B2, vitamin B6, vitaimin B12 and folic acid to a patient in need thereof.

A further aspect of this invention relates to a method of treating or preventing oxazolidinone-associated normocyctic anemia or peripheral sensory neuropathy by administering an effective amount of vitamin B2 to a patient in need thereof.

Yet another aspect of this invention relates to a method of treating or preventing oxazolidinone-associated sideroblastic anemia, peripheral sensory neuropathy, optic neuropathy, seizures, thrombocytopenia, cheilosis, and seborrheic dermatitis by administering an effective amount of vitamin B6 to a patient in need thereof.

Still another aspect of this invention relates to a method of treating or preventing oxazolidinone-associated hypo-regenerative anemia, megaloblastic anemia by administering an effective amount of vitamin B12 and folic acid to a patient in need thereof.

Still another aspect of this invention relates to a method of treating or preventing bacterial infection by administering an effective amount of a compound of formula I and an effective amount of one or more of the group selected from the group consisting of vitamin B2, vitamin B6, vitaimin B12 and folic acid to a patient in need thereof.

The preferred methods of administration of the claimed compositions include oral and parenteral, e.g., i.v. infusion, i.v. bolus and i.m. injection formulated so that a unit dosage comprises a therapeutically effective amount of each active component or some submultiple thereof.

For adults, about 5-50 mg/kg of body weight, preferably about 250 mg to about 1000 mg per person of the cyclopropyl containing oxazolidinone antibacterial compound and about 250 mg, to about 1000 mg per person of the other antbiotic(s) given one to four times daily is preferred. More specifically, for mild infections a dose of about 250 mg two or three times daily of the cyclopropyl containing oxazolidinone antibacterial compound and about 250 mg two or three times daily of the other antibiotic is recommended. For moderate infections against highly susceptible gram positive organisms a dose of about 500 mg each of the cyclopropyl containing oxazolidinone and the other antibiotics, three or four times daily is recommended. For severe, life-threatening infections against organisms at the upper limits of sensitivity to the antibiotic, a dose of about 500-2000 mg each of the cyclopropyl-containing oxazolidinone compound and the other antibiotics, three to four times daily may be recommended.

For children, a dose of about 5-25 mg/kg of body weight given 2, 3, or 4 times per day is preferred; a dose of 10 mg/kg is typically recommended.

The invention is further described in connection with the following non-limiting examples.

### EXAMPLE 1

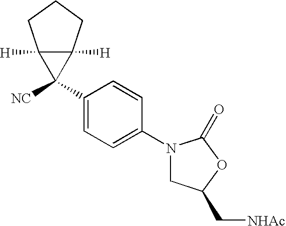

### N-[5(S)-3-[4-[(1α,5α,6β)-(6-Cyanobicyclo[3.1.0]hexan-6-yl)]phenyl]-2-oxooxazolidin-5-ylmethyl]acetamide

Step 1.

### 5(R)-3-[4-[(1α,5α,6β)-(6-Cyanobicyclo[3.1.0]hexan-6-yl)]phenyl]-5-hydroxymethyloxazolidin-2-one

To a solution of 1-benzyloxycarbonylamino-4-[(1α,5α,6β)-(6-cyanobicyclo[3.1.0]hexan-6-yl)]benzene (1.26 g) in dry tetrahydrofuran (25 mL) was added a solution of n-butyllithium in hexane (1.6 M, 2.51 mL) at −78° C., and the mixture was stirred at the same temperature for 30 min. (R)-Glycidyl butyrate (0.58 mL) was added to the mixture at −78° C. and the mixture was stirred at room temperature for 2 hours. After quenching the reaction with the addition of methanol (2.5 mL), the mixture was stirred at room temperature for 30 minutes. After dilution of the mixture with aqueous ammonium chloride solution, the mixture was extracted with ethyl acetate. The organic extracts were washed with brine, dried over anhydrous sodium sulfate, filtered, and then concentrated in vacuo. Flash chromatography (silica, ethyl acetate) of the residue gave 5(R)-3-[4-[(1α,5α,6β)-6-cyanobicyclo[3.1.0]hexan-6-yl)]phenyl]-5-hydroxymethyloxazolidin-2-one (995 mg).

MS (EI⁺) m/z: 298 (M⁺).

HRMS (EI⁺) for C₁₇H₁₈N₂O₃(M⁺): calcd, 298.1317; found, 298.1310.

Step 2.

### 5(R)-Azidomethyl-3-[4-[(1α,5α,6β)-(6-cyanobicyclo[3.1.0]hexan-6-yl)]phenyl]oxazolidin-2-one

To a solution of 5(R)-3-[4-[(1α,5α,6β)-(6-cyanobicyclo[3.1.0]hexan-6-yl)]phenyl]-5-hydroxymethyloxazolidin-2-one (298 mg) in dichloromethane (10 mL) was added triethylamine (0.28 mL) and methanesulfonyl chloride (0.12 mL) at 0° C., the mixture was stirred at the same temperature for 15 minutes. After dilution of the mixture with 1 N hydrochloric acid, the mixture was extracted with ethyl acetate. The organic extracts were washed with water, aqueous sodium hydrogencarbonate solution and brine, dried over anhydrous sodium sulfate, filtered, and then concentrated in vacuo. A suspension of the residue and sodium azide (199 mg) in N,N-dimethylformamide (10 mL) was stirred at 70° C. for 4 hours and concentrated in vacuo. After dilution of the residue with water, the mixture extracted with ethyl acetate. The organic extracts were washed with water and brine, dried over anhydrous sodium sulfate, filtered, and then concentrated in vacuo. Flash chromatography (silica, ethyl acetate) of the residue gave 5(R)-azidomethyl-3-[4-[(1α,5α,6β)-6-cyanobicyclo[3.1.0]hexan-6-yl)]phenyl]oxazolidin-2-one (304 mg).

MS (EI⁺) m/z: 323 (M⁺).

HRMS (EI⁺) for C₁₇H₁₇N₅O₂(M⁺): calcd, 323.1382; found, 323.1363.

Step 3.

### N-[5(S)-3-[4-[(1α,5α,6β)-(6-Cyanobicyclo[3.1.0]hexan-6-yl)]phenyl]-2-oxooxazolidin-5-ylmethyl]acetamide

A suspension of 5(R)-azidomethyl-3-[4-[(1α,5α,6β)-6-cyanobicyclo[3.1.0]hexan-6-yl)]phenyl]oxazolidin-2-one (300 mg) and Lindlar catalyst (5% palladium on CaCO3 partially poisoned with lead, 150 mg) in tetrahydrofuran (2 mL) and methanol (10 mL) was hydrogenated at 1 atm for 70 minutes at room temperature. After filtration of the catalyst, the filtrate was concentrated in vacuo to give 5(R)-aminomethyl-3-[4-[(1α,5α,6β)-6-cyanobicyclo[3.1.0]hexan-yl)]phenyl]oxazolidin-2-one (276 mg). This compound was used without further purification. To a solution of the crude 5(R)-aminomethyl-3-[4-[(1α,5α,6β)-6-cyanobicyclo[3.1.0]hexan-6-yl)]phenyl]oxazolidin-2-one (276 mg) in tetrahydrofuran (10 mL) was added triethylamine (194 μL) and acetic anhydride (108 μL) at 0° C., and the mixture was stirred at room temperature for 30 minutes. After quenching the reaction by the addition of 1 N hydrochloric acid, the mixture was extracted with ethyl acetate. The organic extracts were washed with water, aqueous sodium hydrogencarbonate solution and brine, dried over anhydrous sodium sulfate, filtered, and then concentrated in vacuo. Flash chromatography (silica, ethyl acetate:methanol=15:1) of the residue gave N-[5(S)-3-[4-[(1α,5α,6β)-(6-cyanobicyclo[3.1.0]hexan-6-yl)]phenyl]-2-oxooxazolidin-5-ylmethyl]acetamide (276 mg).

MS (EI⁺) m/z: 339 (M⁺).

HRMS (EI⁺) for C₁₉H₂₁N₃O₃ (M⁺): calcd, 339.1583; found, 339.1606.

### EXAMPLE 2

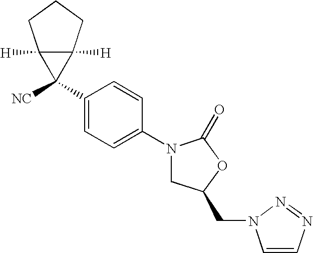

### 1-[5(R)-3-[4-[(1α,5α,6β)-6-Cyanobicyclo[3.1.0]hexan-6-yl)]phenyl]-2-oxooxazolidin-5-ylmethyl]-1,2,3-triazole

The mixture of 5(R)-azidomethyl-3-[4-[(1α,5α,6β)-(6-cyanobicyclo[3.1.0]hexan-6-yl)]phenyl]oxazolidin-2-one (417 mg) and 2,5-norbornadiene (0.70 mL) in dioxane (13 mL) was heated under reflux for 4 hours, and then concentrated in vacuo. Flash chromatography (silica, ethyl acetate:methanol=20:1) of the residue gave 1-[5(R)-3-[4-[(1α,5α,6β)-(6-cyanobicyclo[3.1.0]hexan-6-yl)]phenyl]-2-oxooxazolidin-5-ylmethyl]-1,2,3-triazole (345 mg).

MS (EI⁺) m/z: 349 (M⁺).

HRMS (EI⁺) for C₁₉H₁₉FN₅O₂ (M⁺): calcd, 349.1539; found, 349.1526.

### EXAMPLE 3

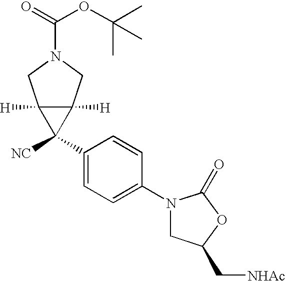

### N-[5(S)-3-[4-[(1α,5α,6β)-(3-t-Butoxycarbonyl-6-cyano-3-azabicyclo[3.1.0]hexan-6-yl)]phenyl]-2-oxooxazolidin-5-ylmethyl]acetamide

Step 1.

### 5(R)-3-[4-[(1α,5α,6β)-3-t-Butoxycarbonyl-6-cyano-3-azabicyclo[3.1.0]hexan-6-yl)]phenyl]-5-hydroxymethyloxazolidin-2-one

The title compound 5(R)-3-[4-[(1α,5α,6β)-(3-t-butoxycarbonyl-6-cyano-3-azabicyclo[3.1.0]hexan-6-yl)]phenyl]-5-hydroxymethyloxazolidin-2-one (102 mg) was prepared from 4-[(1α,5α,6β)-(3-t-butoxycarbonyl-6-cyano-3-azabicyclo[3.1.0]hexan-6-yl)]-1-benzyloxycarbonylaminobenzene (123 mg) in the same manner as described for EXAMPLE 1.

MS (EI⁺) m/z: 399 (M⁺).

HRMS (EI⁺) for C₂₁H₂₅N₃O₅ (M⁺): calcd, 399.1794; found, 399.1801.

Step2.

### N-[5(S)-3-[4-[(1α,5α,6β)-(3-t-Butoxycarbonyl-6-cyano-3-azabicyclo[3.1.0]hexan-6-yl)]phenyl]-2-oxooxazolidin-5-ylmethyl]acetamide

The title compound N-[5(S)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]acetamide (89.9 mg) was prepared from 5(R)-3-[4-[(1α,5α,6β)-3-t-butoxycarbonyl-6-cyano-3-azabicyclo[3.1.0]hexan-6-yl)]phenyl]-5-hydroxymethyloxazolidin-2-one (98.4 mg) in the same manner as described for EXAMPLE 1.

MS (EI⁺) m/z: 440 (M⁺).

HRMS (EI⁺) for C₂₃H₂₈N₄O₅ (M⁺): calcd, 440.2060; found, 440.2076.

### EXAMPLE 4

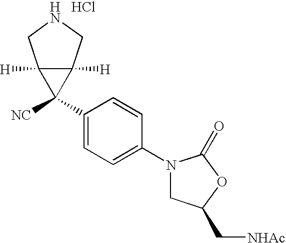

### N-[5(S)-3-[4-[(1α,5α,6β)-(6-Cyano-3-azabicyclo[3.1.0]hexan-yl)]phenyl]-2-oxooxazolidin-5-ylmethyl]acetamide Hydrochloride

To a solution of N-[5(S)-3-[4-[(1α,5α,6β)-3-t-butoxycarbonyl-6-cyano-3-azabicyclo[3.1.0]hexan-6-yl)]phenyl)-2-oxooxazolidin-5-ylmethyl]acetamide (378 mg) in tetrahydrofuran (5 mL) was added a solution of hydrogen chloride in ethanol (10 M, 15 mL) at 0° C., the mixture was stirred at room temperature for 3 hours and concentrated in vacuo. Treatment with ethanol of the residue gave N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (275 mg).

MS (EI⁺) m/z: 340 (M⁺) (as free base).

HRMS (EI⁺) for C₁₈H₂₀N₄O₃ (M⁺): calcd, 340.1535; found, 340.1553.

### EXAMPLE 5

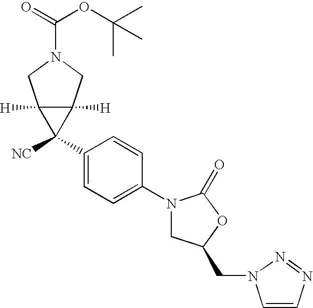

### 1-[5(R)-3-[4-[(1α,5α,6β)-(3-t-Butoxycarbonyl-6-cyano-3-azabicyclo[3.1.0]hexan-6-yl)]phenyl]-2-oxooxazolidin-5-ylmethyl]-1,2,3-triazole

The title compound 1-[5(R)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]-1,2,3-triazole (358 mg) was prepared from 5(R)-azidomethyl-3-[4-[(1α,5α,6β)-(3-t-butoxycarbonyl-6-cyanobicyclo[3.1.0]hexan-6-yl)]phenyl]oxazolidin-2-one (425 mg) in the same manner as described for EXAMPLE 2.

MS (FAB⁺) m/z: 451 (MH⁺).

HRMS (FAB⁺) for C₂₃H₂₇FN₆O₄ (MH⁺): calcd, 451.2094; found, 451.2098.

### EXAMPLE 6

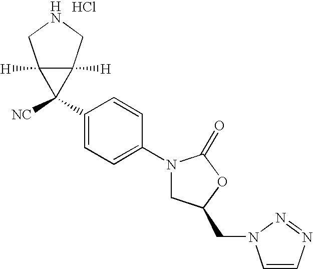

### 1-[5(R)-3-[4-[(1α,5α,6β)-(6-Cyano-3-azabicyclo[3.1.0]hexan-6-yl)]phenyl]-2-oxooxazolidin-5-ylmethyl]-1,2,3-triazole Hydrochloride

1-[5(R)-3-[4-[(1α,5α,6β)-(6-cyano-3-azabicyclo[3.1.0]hexan-6-yl)]phenyl]-2-oxooxazolidin-5-ylmethyl]-1,2,3-triazole hydrochloride (267 mg) was prepared from 1-[5(R)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]-1,2,3-triazole (358 mg) in the same manner as described for EXAMPLE 4.

MS (EI⁺) m/z: 350 (M⁺) (as free base).

HRMS (EI⁺) for C₁₈H₁₈N₆O₂(M⁺): calcd, 350.1491; found, 350.1464.

### EXAMPLE 7

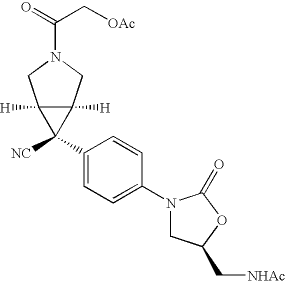

### N-[5(S)-3-[4-[(1α,5α,6β)-(3-Acetoxyacetyl-6-cyano-3-azabicyclo[3.1.0]hexan-6-yl)]phenyl]-2-oxooxazolidin-5-ylmethyl]acetamide

To a suspension of N-[5(S)-3-[4-[(1α,5α,6β)-6-cyano-3-azabicyclo[3.1.0]hexan-6-yl)]phenyl]-2-oxooxazolidin-5-ylmethyl]acetamide hydrochloride (415 mg) in dichloromethane (11 mL) was added triethylamine (0.46 mL) and acetoxyacetyl chloride (0.15 mL) at 0° C., the mixture was stirred at the same temperature for 45 minutes. After dilution of the mixture with water, the mixture was extracted with dichloromethane. The organic extracts were dried over anhydrous sodium sulfate, filtered, and then concentrated in vacuo. Flash chromatography (silica, dichloromethane:methanol=10:1) of the residue gave N-[5(S)-3-[4-[(1α,5α,6β)-(3-acetoxyacetyl-6-cyano-3-azabicyclo[3.1.0]hexan-6-yl)]phenyl]-2-oxooxazolidin-5-ylmethyl]acetamide (378 mg).

MS (FAB⁺) m/z: 441 (MH⁺).

HRMS (FAB⁺) for C₂₂H₂₅N₄O₆ (MH⁺): calcd, 441.1774; found, 441.1764.

### EXAMPLE 8

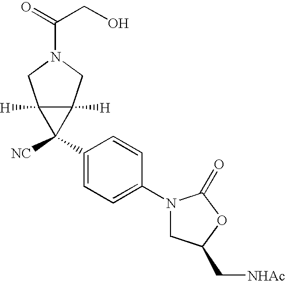

### N-[5(S)-3-[4-[(1α,5α,6β)-(6-Cyano-3-hydroxyacetyl-3-azabicyclo[3.1.0]hexan-6-yl)]phenyl]-2-oxooxazolidin-5-ylmethyl]acetamide

To a suspension of N-[5(S)-3-[4-[(1α,5α,6β)-(3-acetoxyacetyl-6-cyano-3-azabicyclo[3.1.0]hexan-6-yl)]phenyl]-2-oxooxazolidin-5-ylmethyl]acetamide (225 mg) in methanol (5 mL) and tetrahydrofuran (1 mL) was added potassium carbonate (141 mg) at room temperature, the mixture was stirred at the same temperature for 90 minutes and concentrated in vacuo. Flash chromatography (silica, dichloromethane:methanol=20:1) of the residue gave N-[5(S)-3-[4-[(1α,5α,6β)-(6-cyano-3-hydroxyacetyl-3-azabicyclo[3.1.0]hexan-6-yl)]phenyl]-2-oxooxazolidin-5-ylmethyl]acetamide (138 mg).

MS (FAB⁺) m/z: 399 (MH⁺).

HRMS (FAB⁺) for C₂₀H₂₃N₄O₅ (MH⁺): calcd, 399.1668; found, 399.1681.

### EXAMPLE 9

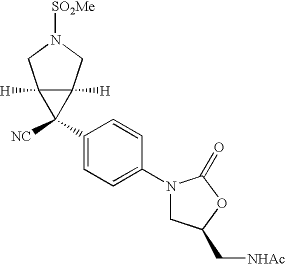

### N-[5(S)-3-[4-[(1α,5α,6β)-(6-Cyano-3-methanesulfonyl-3-azabicyclo[3.1.0]hexan-6-yl)]phenyl]-2-oxooxazolidin-5-ylmethyl]acetamide

The title compound N-[5(S)-3-[4-[(1α,5α,6β)-(6-cyano-3-methanesulfonyl-3-azabicyclo[3.1.0]hexan-6-yl)]phenyl]-2-oxooxazolidin-5-ylmethyl]acetamide (219 mg) was prepared from N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (226 mg) and methanesulfonyl chloride (70 μL) in the same manner as described for EXAMPLE 7.

MS (FAB⁺) m/z: 419 (MH⁺).

HRMS (FAB⁺) for C₁₉H₂₃N₄O₅S (MH⁺): calcd, 419.1389; found, 419.1386.

### EXAMPLE 10

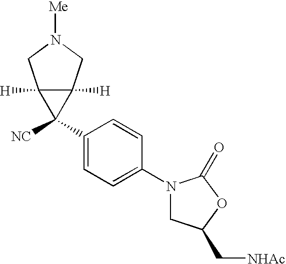

### N-[5(S)-3-[4-[(1α,5α,6β6-Cyano-3-methyl-3-azabicyclo[3.1.0]hexan-6-yl)]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (188 mg) in tetrahydrofuran (5 mL) was added acetic acid (57 μL), 35% formaldehyde (396 μL), and sodium triacetoxyborohydride (223 m) at room temperature, the mixture was stirred at the same temperature for 2 hours. After quenching the reaction by addition of aqueous sodium hydrogencarbonate solution, the mixture was extracted with dichloromethane-methanol (5:1). The organic extracts were dried over anhydrous sodium sulfate, filtered, and then concentrated in vacuo. Flash chromatography (silica, dichloromethane:methanol=10:1) of the residue gave N-[5(S)-3-[4-[(1α,5α,6β)-(6-cyano-3-methyl-3-azabicyclo[3.1.0]hexan-6-yl)]phenyl]-2-oxooxazolidin-5-ylmethyl]acetamide (104 mg).

MS (FAB⁺) m/z: 355 (MH⁺).

HRMS (FAB⁺) for C₁₉H₂₃N₄O₃ (MH⁺): calcd, 355.1770; found, 355.1775.

### EXAMPLE 11

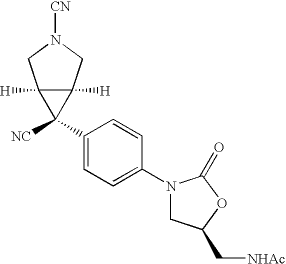

### N-[5(S)-3-[4-[(1α,5α,6β)-(3,6-Dicyano-3-azabicyclo[3.1.0]hexan-6-yl)]phenyl]-2-oxooxazolidin-5-ylmethyl]acetamide

A suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (245 mg) and sodium acetate (373 mg) in methanol (22 mL) was stirred at room temperature for 20 minutes. To the resulting suspension was added a solution of cyanogens bromide in dichloromethane (5 M, 0.26 mL) at 0° C., the mixture was stirred at the same temperature for 40 minutes, and concentrated in vacuo. Flash chromatography (silica, dichloromethane:methanol=10:1) of the residue gave N-[5(S)-3-[4-[(1α,5α,6β)-3,6-dicyano-3-azabicyclo[3.1.0]hexan-6-yl)]phenyl]-2-oxooxazolidin-5-ylmethyl]acetamide (207 mg).

MS (FAB⁺) m/z: 366 (MH⁺).

HRMS (FAB⁺) for C₁₉H₂₀N₅O₃ (MH⁺): calcd, 366.1566; found, 366.1575.

### EXAMPLE 12

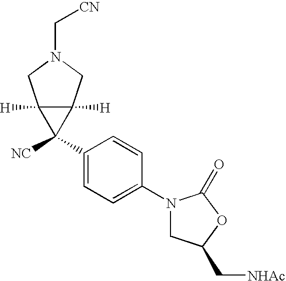

### N-[5(S)-3-[4-[(1α,5α,6β)-(6-Cyano-3-cyanomethyl-3-azabicyclo[3.1.0]hexan-6-yl)]phenyl]-2-oxooxazolidin-5-ylmethyl]acetamide

A suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (245 mg), sodium hydrogencarbonate (273 mg) in N,N-dimethylformamide (6.5 mL) was stirred at room temperature for 10 minutes. To the resulting suspension was added bromoacetonitrile (70 μL) at room temperature, the mixture was stirred at the same temperature for 6 hours, and concentrated in vacuo. Flash chromatography (silica, dichloromethane:methanol=10:1) of the residue gave N-[5(S)-3-[4-[(1α,5α,6β)-(6-cyano-3-cyanomethyl-3-azabicyclo[3.1.0]hexan-6-yl)]phenyl]-2-oxooxazolidin-5-ylmethyl]acetamide (219 mg).

MS (FAB⁺) m/z: 380 (MH⁺).

HRMS (FAB⁺) for C₂₀H₂₂N₅O₃ (MH⁺): calcd, 380.1723; found, 380.1728.

### EXAMPLE 13

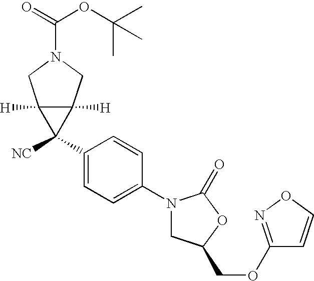

### 5(R)-3-[4-[(1α,5α,6β)-(3-t-Butoxycarbonyl-6-cyano-3-azabicyclo[3.1.0]hexan-6-yl)]phenyl]-5-[(isoxazol-3-yl)oxy]methyloxazolidin-2-one

To a suspension of 5(R)-3-[4-[(1α,5α,6β)-(3-t-butoxycarbonyl-6-cyano-3-azabicyclo[3.1.0]hexan-6-yl)]phenyl]-5-hydroxymethyloxazolidin-2-one (10.0 mg), 3-hydroxyisoxazole (4.3 mg) and triphenylphosphine (13.5 mg) in tetrahydrofuran (0.25 mL) was added diisopropyl azodicarboxylate (9.8 μL), the mixture was stirred at room temperature for 3 hours, and concentrated in vacuo. Flash chromatography (silica, hexane:ethyl acetate=1:5) of the residue gave 5(R)-3-[4-[(1α,5α,6β)-(3-t-butoxycarbonyl-6-cyano-3-azabicyclo[3.1.0]hexan-yl)]phenyl]-5-[(isoxazol-3-yl)oxy]methyloxazolidin-2-one (11.7 mg).

MS (FAB⁺) m/z: 467 (MH⁺).

HRMS (FAB⁺) for C₂₄H₂₇N₄O₆ (MH⁺): calcd, 467.1931; found, 467.1903.

### EXAMPLE 14

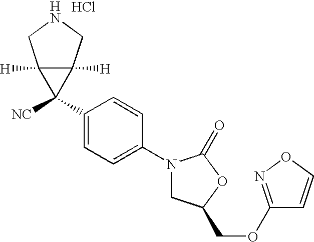

### 5(R)-3-[4-[(1α,5α,6β)-(6-Cyano-3-azabicyclo[3.1.0]hexan-6-yl)]phenyl]-5-[(isoxazolyl-3-yl)oxy]methyloxazolidin-2-one Hydrochloride

The title compound 5(R)-3-[4-[(1α,5α,6β)-6-cyano-3-azabicyclo[3.1.0]hexan-yl)]phenyl]-5-[(isoxazolyl-3-yl)oxy]methyloxazolidin-2-one hydrochloride (199 mg) was prepared from 5(R)-3-[4-[(1α,5α,6β)-(3-t-butoxycarbonyl-6-cyano-3-azabicyclo[3.1.0]hexan-6-yl)]phenyl]-5-[(isoxazolyl-3-yl)oxy]methyloxazolidin-2-one (248 mg) in the same manner as described for EXAMPLE 4.

MS (EI⁺) m/z: 366 (M⁺) (as free base).

HRMS (EI⁺) for C₁₉H₁₈N₄O₄ (M⁺): calcd, 366.1328; found, 366.1330.

### EXAMPLE 15

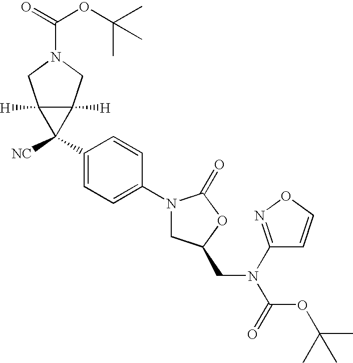

### 5(R)-3-[4-[(1α,5α,6β)-(3-t-Butoxycarbonyl-6-cyano-3-azabicyclo[3.1.0]hexan-6-yl)]phenyl]-5-[N-(t-butoxycarbonyl)-N-(isoxazol-3-yl)]aminomethyloxazolidin-2-one

To a suspension of 5(R)-3-[4-[(1α,5α,6β)-(3-t-butoxycarbonyl-6-cyano-3-azabicyclo[3.1.0]hexan-6-yl)]phenyl]-5-hydroxymethyloxazolidin-2-one (10.0 mg), 3-N-(t-butoxycarbonyl)aminoisoxazole (9.2 mg), and tetramethylazodicarboxamide (8.6 mg) in benzene (0.25 mL) was added tributylphosphine (12.5 μL), and the mixture was stirred at room temperature for 90 minutes. After dilution of the mixture with ethyl acetate, the insoluble materials were filtered off, and the filtrate was concentrated in vacuo. Flash chromatography (silica, hexane:ethyl acetate=3:5) of the mixture gave 5(R)-3-[4-[(1α,5α,6β)-(3-t-butoxycarbonyl-6-cyano-3-azabicyclo[3.1.0]hexan-6-yl)]phenyl]-5-[N-(t-butoxycarbonyl)-N-(isoxazol-3-yl)]aminomethyloxazolidin-2-one (14.1 mg).

MS (FAB⁺) m/z: 566 (MH⁺).

HRMS (FAB⁺) for C₂₉H₃₆N₅O₇ (MH⁺): calcd, 566.2615; found, 566.2609.

### EXAMPLE 16

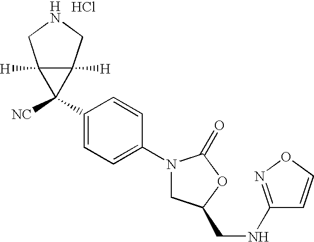

### 5(R)-3-[4-[(1α,5α,6β)-(6-Cyano-3-azabicyclo[3.1.0]hexan-6-yl)]phenyl]-5-[N-(isoxazol-3-yl)]aminomethyloxazolidin-2-one Hydrochloride

The tide compound 5(R)-3-[4-[(1α,5α,6β)-6-cyano-3-azabicyclo[3.1.0]hexan-6-yl)]phenyl]-5-[N-isoxazol-3-yl)]aminomethyloxazolidin-2-one hydrochloride (207 mg) was prepared from (R)-3-[4-[(1α,5α,6β)-3-t-butoxycarbonyl-6-cyano-3-azabicyclo[3.1.0]hexan-6-yl)]phenyl]-5-[N-t-butoxycarbonyl)-N-(isoxazol-3-yl)]aminomethyloxazolidin-2-one (292 mg) in the same manner as described for EXAMPLE 4.

MS (EI⁺) m/z: 365 (M⁺) (as free base).

HRMS (EI⁺) for C₁₉H₁₉N₅O₃ (M⁺): calcd, 365.1488; found, 365.1478.

### EXAMPLE 17

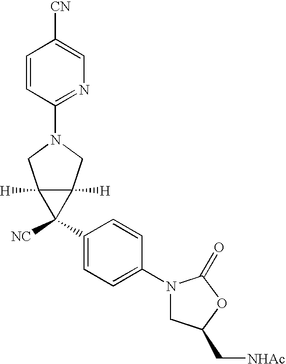

### N-[5(S)-3-[4-[(1α,5α,6β)-[6-Cyano-3-5-cyanopyridin-2-yl)-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (226 mg) in dimethyl sulfoxide (6 mL) was added diisopropylethylamine (1.05 mL), the mixture was stirred at room temperature for 5 minutes. To the resulting mixture was added 2-chloro-5-cyanopyridine (166 mg), the mixture was stirred at 40° C. for overnight, and stirred at 60° C. for 10 hours. After dilution of the mixture with ethyl acetate and water, the mixture was extracted with etyl acetate. The organic extracts were washed with aqueous sodium hydrogencarbonate solution and brine, dried over anhydrous sodium sulfate, filtered, and then concentrated in vacuo. Flash chromatography (silica, dichloromethane:methanol=10:1) of the residue gave N-[5(S)-3-[4-[(1α,5α,6β) -[6-cyano-3-5-cyanopyridin-2-yl)-3-azabicyclo[3.1.0]hexan-6-yl]]phenyl]-2-oxooxazolidin-5-ylmethyl]acetamide (218 mg).

MS (FAB⁺) m/z: 443 (MH⁺).

HRMS (FAB⁺) for C₂₄H₂₃N₆O₃ (MH⁺): calcd, 443.1832; found, 443.1841.

### EXAMPLE 18

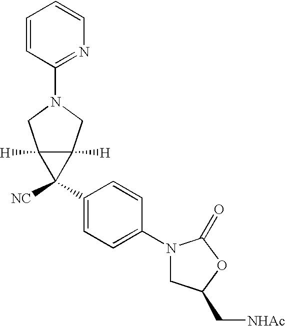

### N-[5(S)-3-[4-[(1α,5α,6β)-[6-Cyano-3-(pyridin-2-yl)-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (264 mg) in 2-pyridyl trifluoromethanesulfonate (5.49 mL) was added diisopropylethylamine (1.22 mL), the mixture was stirred at room temperature for 5 minutes, and stirred at 90° C. for 30.5 hours. Flash chromatography (silica, ethyl acetate:methanol=5:1) of the mixture gave N-[5(S)-3-[4-[(1α,5α,6β)-[6-cyano-3-(pyridin-2-yl)-3-azabicyclo[3.1.0]hexan-6-yl]]phenyl]-2-oxooxazolidin-5-ylmethyl]acetamide (215 mg).

MS (FAB⁺) m/z: 418 (MH⁺).

HRMS (FAB⁺) for C₂₃H₂₄N₅O₃ (MH⁺): calcd, 418.1879; found, 418.1885.

### EXAMPLE 19

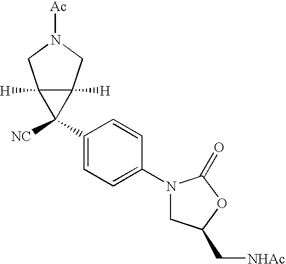

### N-[5(S)-3-[4-[(1α,5α,6β)-[3-Acetyl-6-cyano-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (245 mg) in tetrahydrofuran (6.5 mL) was added saturated sodium hydrogencarbonate solution (6.5 mL), the mixture was stirred at 0° C. for 5 minutes. To the resulting mixture was added acetic anhydride (70 μL), the mixture was and stirred at 0° C. for 20 minutes. After an aqueous layer of the mixture was extracted with dicloromethane-methanol (10:1), the combined organic extracts were dried over anhydrous sodium sulfate, filtered, and then concentrated in vacuo. Treatment of the residue with ethyl acetate gave N-[5(S)-3-[4[(1α,5α,6β)-[3-acetyl-6-cyano-3-azabicyclo[3.1.0]hexan-6yl]]phenyl]-2-oxooxazolidin-5-ylmethyl]acetamide (215 mg).

MS (FAB⁺) m/z: 383 (MH⁺).

HRMS (FAB⁺) for C₂₀H₂₃N₄O₄ (MH⁺): calcd, 383.1719; found, 383.1732.

### EXAMPLE 20

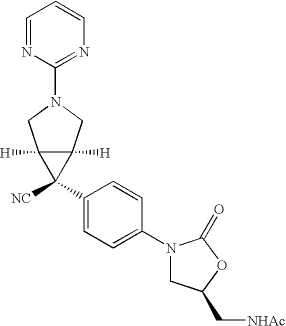

### N-[5(S)-3-[4-[(1α,5α,6β)-[6-Cyano-3-(pyrimidin-2-yl)-3-azabicyclo[3.1.0]hexan-6-yl]]phenyl]-2-oxooxazolidin-5-ylmethyl]acetamide

The title compound N-[5(S)-3-[4-[(1α,5α,6β)-[6-cyano-3-pyrimidin-2-yl)-3-azabicyclo[3.1.0]hexan-6-yl]]phenyl]-2-oxooxazolidin-5-ylmethyl]acetamide (198 mg) was prepared from N-[5(S)-3-[4-[(1α,5α,6β)-6-cyano-3-azabicyclo[3.1.0]hexan-6-yl)]phenyl]-2-oxooxazolidin-5-ylmethyl]acetamide hydrochloride (283 mg) and 2-chloropyrimidine (181 mg) in the same manner as described for EXAMPLE 17.

MS (FAB⁺) m/z: 419 (MH⁺).

HRMS (FAB⁺) for C₂₂H₂₃N₆O₃ (MH⁺): calcd, 419.1832; found, 419.1832.

### EXAMPLE 21

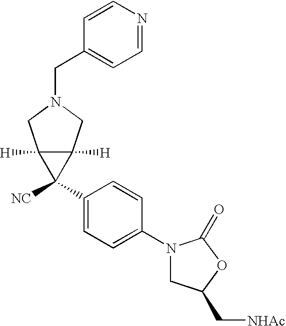

### N-[5(S)-3-[4-[(1α,5α,6β)-[6-Cyano-3-(4-pyridylmethyl)-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (188 mg) in dichloromethane (33 mL) was added triethylamine (209 μL), the mixture was stirred at room temperature for 5 minutes. To the resulting mixture was added pyridine 4-carboxamide (98 μL), acetic acid (115 μL), and sodium triacetoxyborohydride (223 mg) at room temperature, the mixture was stirred at room temperature for 7 hours. After quenching the reaction by addition of 1N sodium hydroxide solution, the mixture was extracted with dichloromethane. The organic extracts were dried over anhydrous sodium sulfate, filtered, and then concentrated in vacuo. Flash chromatography (silica, dichloromethane:methanol=10:1) of the residue gave N-[5(S)-3-[4-[(1α,5α,6β)-[6-cyano-3-4-pyridylmethyl)-3-azabicyclo[3.1.0]hexan-6-yl]]phenyl]-2-oxooxazolidin-5-ylmethyl]acetamide (181 mg).

MS (FAB⁺) m/z: 432 (MH⁺).

HRMS (FAB⁺) for C₂₄H₂₆N₅O₃ (MH⁺): calcd, 432.2036; found, 432.2041.

### EXAMPLE 22

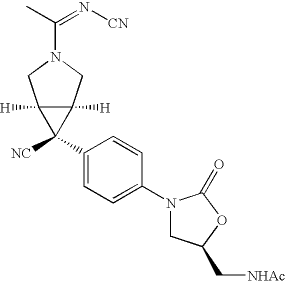

### N-[5(S)-3-[4-[(1α,5α,6β)-[6-Cyano-3-(N-cyano-1-iminoethyl)-3-azabicyclo[3.1.0]hexan-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (207 mg) in methanol (5.5 mL) was added diisopropylethylamine (192 μL), the mixture was stirred at room temperature for 20 minutes. To the resulting mixture was added methyl N-cyanoacetoimidate (108 mg), the mixture was stirred at room temperature for 2 days. The resulting precipitates were collected by filtration to give N-[5(S)-3-[4-[(1α,5α,6β)-[6-cyano-3-(N-cyano-1-iminoethyl)-3-azabicyclo[3.1.0]hexan-6-yl]]phenyl]-2-oxooxazolidin-5-ylmethyl]acetamide (186 mg).

MS (FAB⁺) m/z: 407 (MH⁺).

HRMS (FAB⁺) for C₂₁H₂₃N₆O₃ (MH⁺): calcd, 407.1832; found, 407.1869.

### EXAMPLE 23

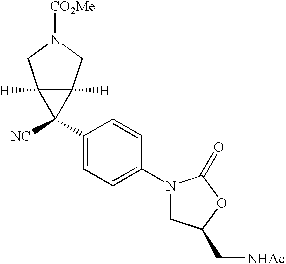

### N-[5(S)-3-[4[(1α,5α,6β)-[6-Cyano-3-methoxycarbonyl-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (188 mg) in acetonitrile (5 mL) was added diisopropylethylamine (261 μL), the mixture was stirred at room temperature for 10 minutes. To the resulting mixture was added methyl chloroformate (61 μL) at 0° C., the mixture was stirred at room temperature for 25 minutes, and concentrated in vacuo. After dilution of the residue with 1N hydrochloric acid, the mixture was extracted with dichloromethane. The organic extracts were dried over anhydrous sodium sulfate, filtered, and then concentrated in vacuo. Treatment of the residue with ethyl acetate gave N-[5(S)-3-[4-[(1α,5α,6β)-[6-cyano-3-methoxycarbonyl-3-azabicyclo[3.1.0]hexan-6-yl]]phenyl]-2-oxooxazolidin-5-ylmethyl]acetamide (161 mg).

MS (FAB⁺) m/z: 399 (MH⁺).

HRMS (FAB⁺) for C₂₀H₂₃N₄O₅(MH⁺): calcd, 399.1668; found, 399.1671.

### EXAMPLE 24

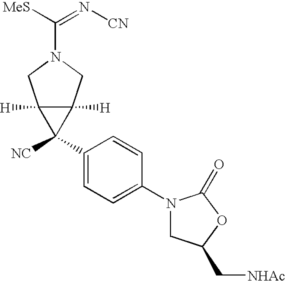

### N-[5(S)-3-[4-[(1α,5α,6β)-[6-Cyano-3-N-cyano-S-methylthioiminomethyl)-3-azabicyclo[3.1.0]hexan-6-yl]]phenyl]-2-oxooxazolidin-5-ylmethyl]acetamide

The title compound N-[5(S)-3-[4-[(1α,5α,6β)-[6-cyano-3-N-cyano-S-methylthioiminomethyl)-3-azabicyclo[3.1.0]hexan-6-yl]]phenyl]-2-oxooxazolidin-5-ylmethyl]acetamide (8.3 mg) was prepared from N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (9.4 mg) and dimethyl N-cyanodithioiminocarbonate (4.9 mg) in the same manner as described for EXAMPLE 11.

MS (FAB⁺) m/z: 439 (MH⁺).

HRMS (FAB⁺) for C₂₁H₂₃N₆O₃S (MH⁺): calcd, 439.1552; found, 439.1553.

### EXAMPLE 25

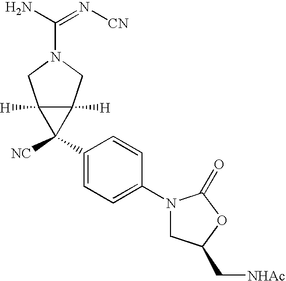

### N-[5(S)-3-[4-[(1α,5α,6β)-[6-Cyano-3-(N-cyanocarboxamidyl)-3-azabicyclo[3.1.0]hexan-6-yl]]phenyl]-2-oxooxazolidin-5-ylmethyl]acetamide

To a solution of N-[5(S)-3-[4-[(1α,5α,6β)-[6-cyano-3-(N-cyano-S-methylthioiminomethyl)-3-azabicyclo[3.1.0]hexan-6-yl]]phenyl]-2-oxooxazolidin-5-ylmethyl]acetamide (260 mg) in dimethylformamide (8 mL) was added a solution of ammonia in methanol (4.1 M, 8 mL) at 0° C., the mixture was allowed stand at room temperature for 3 days, and concentrated in vacuo. Flash chromatography (silica, dichloromethane:methanol=20:3) of the residue gave N-[5(S)-3-[4-[(1α,5α,6β)-[6-cyano-3-N-cyanocarboxamidyl)-3-azabicyclo[3.1.0]hexan-6-yl]]phenyl]-2-oxooxazolidin-5-ylmethyl]acetamide (111 mg).

MS (FAB⁺) m/z: 408 (MH⁺).

HRMS (FAB⁺) for C₂₀H₂₂N₇O₃ (MH⁺): calcd, 408.1784; found, 408.1792.

### EXAMPLE 26

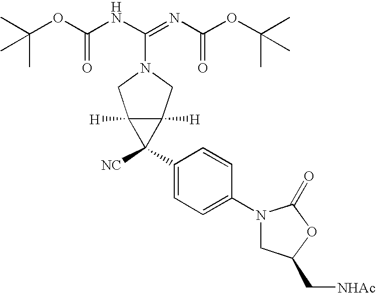

### N-[5(S)-3-[4-[(1α,5α,6β)-[3 (N,N′-t-Butoxycarbonylcarboxamidyl)-6-cyano-3-azabicyclo[3.1.0]hexan-6-yl]]phenyl]-2-oxooxazolidin-5-ylmethyl]acetamide

The title compound N-[5(S)-3-[4-[(1α,5α,6β)-[3-N,N′-t-butoxycarbonylcarboxamidyl)-6-cyano-3-azabicyclo[3.1.0]hexan-6-yl]]phenyl]-2-oxooxazolidin-5-ylmethyl]acetamide (35 mg) was prepared from N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (19 mg) and N,N′-di(t-butoxycarbonyl)-1H-pyrazole-1-carboxamidine (23 mg) in the same manner as described for EXAMPLE 23.

MS (FAB⁺) m/z: 583 (MH⁺).

HRMS (FAB⁺) for C₂₉H₃₉N₆O₇ (MH⁺): calcd, 583.2880; found, 583.2880.

### EXAMPLE 27

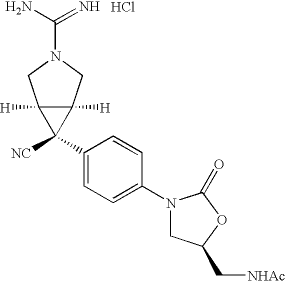

### N-[5(S)-3-[4-[(1α,5α,6β)-[3-Carboxamidyl-6-cyano-3-azabicyclo[3.1.0]hexan-6-yl]]phenyl]-2-oxooxazolidin-5-ylmethyl]acetamide Hydrochloride

To a suspension of N-[5(S)-3-[4-[(1α,5α,6β)-[3-(N,N′-t-butoxycarbonylcarboxamidyl)-6-cyano-3-azabicyclo[3.1.0]hexan-6-yl]]phenyl]-2-oxooxazolidin-5-ylmethyl]acetamide (221 mg) in tetrahydrofuran (1.9 mL) was added a solution of hydrogen chloride in dioxane (4.8 M, 5.7 mL) at 0° C., the mixture was stirred at room temperature for 3.7 hours, and concentrated in vacuo. After dilution of the residue with water, the mixture was washed with ethyl acetate. The resulting aqueous layer was lyophilized to give N-[5(S)-3-[4-[(1α,5α,6β)-[3-carboxamidyl-6-cyano-3-azabicyclo[3.1.0]hexan-6-yl]]phenyl]-2-oxooxazolidin-5-ylmethyl]acetamide hydrochloride (157 mg).

MS (FAB⁺) m/z: 383 (MH⁺) (as free base).

HRMS (FAB⁺) for C₁₉H₂₃N₆O₃ (MH⁺): calcd, 383.1832; found, 383.1879.

### EXAMPLE 28

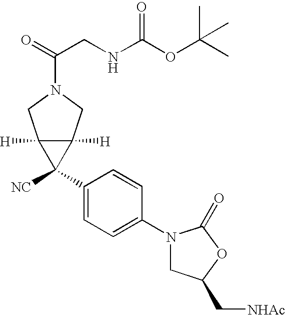

### N-[5(S)-3-[4-[(1α,5α,6β)-[3-(N-t-Butoxycarbonylamino)acetyl-6-cyano-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (19 mg), N-t-butoxycarbonylglycine (9.6 mg), and 1-hydroxybenzotriazole (8.4 mg) in dimethylformamide (2 mL) was added triethylamine (17 μL), the mixture was stirred at room temperature for 5 minutes. T the resulting mixture was added 1-(3-dimethylaminopropyl)-3-ethylcarbodiimide hydrochloride (11 mg) at 0° C., the mixture was stirred at room temperature for 5.5 hours, and concentrated in vacuo. After dilution of the residue with 1N hydrochloric acid, the mixture was extracted with ethyl acetate. The organic extracts were washed with aqueous sodium hydrogencarbonate solution, dried over anhydrous sodium sulfate, filtered, and then concentrated in vacuo. Flash chromatography (silica, ethyl acetate:methanol=5:1) of the residue gave N-[5(S)-3-[4-[(1α,5α,6β)-[3-N-t-Butoxycarbonylamino)acetyl-6-cyano-3-azabicyclo[3.1.0]hexan-6-yl]]phenyl]-2-oxooxazolidin-5-ylmethyl]acetamide (23 mg).

MS (FAB⁺) m/z: 498 (MH⁺).

HRMS (FAB⁺) for C₂₅H₃₂N₅O₆ (MH⁺): calcd, 498.2353; found, 498.2339.

### EXAMPLE 29

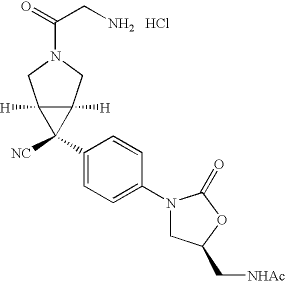

### N-[5(S)-3-[4-[(1α,5α,6β)-[3-Aminoacetyl-6-cyano-3-azabicyclo[3.1.0]hexan-6-yl]]phenyl]-2-oxooxazolidin-5-ylmethyl]acetamide Hydrochloride

The title compound N-[5(S)-3-[4-[(1α,5α,6β)-[3-aminoacetyl-6-cyano-3-azabicyclo[3.1.0]hexan-6-yl]]phenyl]-2-oxooxazolidin-5-ylmethyl]acetamide hydrochloride (225 mg) was prepared from N-[5(S)-3-[4-[(1α,5α,6β)-[3-(N-t-butoxycarbonylamino)acetyl-6-cyano-3-azabicyclo[3.1.0]hexan-6-yl]]phenyl]-2-oxooxazolidin-5-ylmethyl]acetamide (299 mg) in the same manner as described for EXAMPLE 27.

MS (FAB⁺) m/z: 398 (MH⁺) (as free base).

HRMS (FAB⁺) for C₂₀H₂₄N₅O₄ (MH⁺): calcd, 398.1828; found, 398.1826.

### EXAMPLE 30

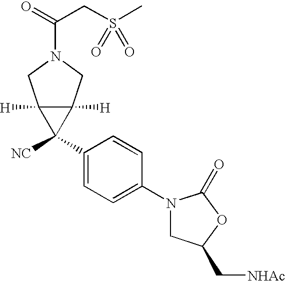

### N-[5(S)-3-[4-[(1α,5α,6β)-[6-Cyano-3-methanesulfonylacetyl-3-azabicyclo[3.1.0]hexan-6-yl]]phenyl]-2-oxooxazolidin-5-ylmethyl]acetamide

The title compound N-[5(S)-3-[4-[(1α,5α,6β)-[6-cyano-3-methanesulfonylacetyl-3-azabicyclo[3.1.0]hexan-6-yl]]phenyl]-2-oxooxazolidin-5-ylmethyl]acetamide (193 mg) was prepared from N-[5(S)-3-[4-[(1α,5α,6β)-(6-cyano-3-azabicyclo[3.1.0]hexan-6-yl)]phenyl]-2-oxooxazolidin-5-ylmethyl]acetamide hydrochloride (188 mg) and methanesulfonylacetic acid (78 mg) in the same manner as described for EXAMPLE 28.

MS (FAB⁺) m/z: 461 (MH⁺).

HRMS (FAB⁺) for C₂₁H₂₅N₄O₆S (MH⁺): calcd, 461.1495; found, 461.1513.

### EXAMPLE 31

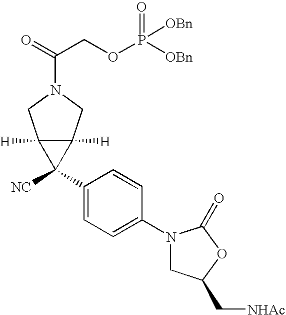

### N-[5(S)-3-[4-[(1α,5α,6β)-[6-Cyano-3-(dibenzylphosphoryloxy)acetyl-3-azabicyclo[3.1.0]hexan-6-yl]]phenyl]-2-oxooxazolidin-5-ylmethyl]acetamide

To a suspension of N-[5(S)-3-[4-[(1α,5α,6β)-(6-cyano-3-hydroxyacetyl-3-azabicyclo[3.1.0]hexan-6-yl)]phenyl]-2-oxooxazolidin-5-ylmethyl]acetamide (10 mg), triphenylphosphine (14 mg), and dibenzyl phosphate (14 mg) in tetrahydrofuran (1 mL) was added an solution of diisopropyl azodicarboxylate in toluene (40 wt %, 27 μL), the mixture was stirred at room temperature for overnight, and stirred at 60° C. for 11 hours. The mixture was concentrated in vacuo. Flash chromatography (silica, dichloromethane:methanol=20:3) of the residue gave N-[5(S)-3-[4-[(1α,5α,6β)-[6-cyano-3-(dibenzylphosphoryloxy)acetyl-3-azabicyclo[3.1.0]hexan-6-yl]]phenyl]-2-oxooxazolidin-5-ylmethyl]acetamide (9.5 mg).

MS (FAB⁺) m/z: 659 (MH⁺).

HRMS (FAB⁺) for C₃₄H₃₆N₄O₈P (MH⁺): calcd, 659.2271; found, 659.2256.

### EXAMPLE 32

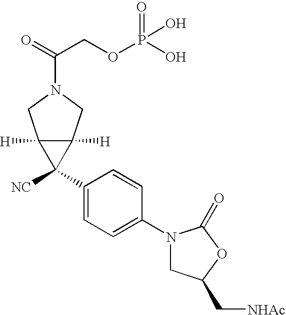

### N-[5(S)-3-[4-[(1α,5α,6β)-[6-Cyano-3-phosphoryloxy)acetyl-3-azabicyclo[3.1.0]hexan-6-yl]]phenyl]-2-oxooxazolidin-5-ylmethyl]acetamide

A suspension of N-[5(S)-3-[4-[(1α,5α,6β)-[6-cyano-3-(dibenzylphosphoryloxy)acetyl-3-azabicyclo[3.1.0]hexan-6-yl]]phenyl]-2-oxooxazolidin-5-ylmethyl]acetamide (163 mg) and palladium on charcoal (7.5%,24 mg) in methanol (6 mL) was stirred at room temperature for 4 hours under hydrogen atmosphere. After insoluble materials were filtered off, the filtrate was concentrated in vacuo. A solution of the residue in water (1.5 mL) was washed with ethyl acetate, the resulting aqueous solution was lyophilized to give N-[5(S)-3-[4-[(1α,5α,6β)-[6-cyano-3-phosphoryloxy)acetyl-3-azabicyclo[3.1.0]hexan-6-yl]]phenyl]-2-oxooxazolidin-5-ylmethyl]acetamide (111 mg).

MS (FAB⁺) m/z: 479 (MH⁺).

HRMS (FAB⁺) for C₂₀H₂₄N₄O₈P (MH⁺): calcd, 479.1332; found, 479.1344.

### REFERENCE EXAMPLE 1

### 4-[(1α,5α,6β)-(6-Cyanobicyclo[3.1.0]hexan-6-yl)]-1-benzyloxycarbonylaminobenzene

Step 1.

### (1α,5α,6β)-(6-Cyanobicyclo[3.1.0]hexan-6-yl)benzene

To a solution of lithium diisopropylamide (prepared from diisopropylamine (3.88 mL) and n-butyllithium (1.6 M solution in hexane, 17.4 mL)) in tetahydrofuran (37 mL) was added phenylacetonitrile (3.18 mL) at −50° C., the mixture was stirred at 0° C. for 3 hours. To the mixture was added a solution of cyclopenten-1-yl phenyl sulfone (5.49 g) in tetrahydrofuran (26 mL) at 5° C., the mixture was stirred at the same temperature for 40 minutes, and stirred at room temperature for 18 hours. The mixture was stirred at 60° C. for 3 hours. After dilution of the mixture with aqueous ammonium chloride solution, the mixture was extracted with ethyl acetate. The organic extracts were washed with brine, dried over anhydrous sodium sulfate, filtered, and then concentrated in vacuo. Flash chromatography (silica, hexane:ethyl acetate=10:1) of the residue gave (1α,5α,6β)-(6-cyanobicyclo[3.1.0]hexan-6-yl)benzene (4.44 g).

MS (EI⁺) m/z: 183 (M⁺).

HRMS (EI⁺) for C₁₃H₁₃N (M⁺): calcd, 183.1048; found, 183.1072.

Step 2.

### 4-(1α,5α,6β)-(6-cyanobicyclo[3.1.0]hexan-6-yl)-1-nitrobenzene

To a solution of (1α,5α,6β)-(6-cyanobicyclo[3.1.0]hexan-6-yl)benzene (916 mg) in chloroform (5 mL) was added concentrated sulfuric acid (1.93 mL) and nitric acid (fuming, 0.28 mL) at −30° C., the mixture was stirred at the same temperature for 1 minute. The mixture was poured into ice water, extracted with chloroform. The organic extracts were washed with aqueous sodium hydrogencarbonate solution and brine, dried over anhydrous sodium sulfate, filtered, and then concentrated in vacuo. Flash chromatography (silica, hexane:ethyl acetate=10:3) of the residue gave 4-(1α,5α,6β)-(6-cyanobicyclo[3.1.0]hexan-6-yl)-1-nitrobenzene (875 mg).

MS (EI⁺) m/z: 228 (M⁺).

HRMS (EI⁺) for C₁₃H₁₂N₂O₂ (M⁺): calcd, 228.0899; found, 228.0889.

Step 3.

### 1-Benzyloxycarbonylamino-4-[(1α,5α,6β)-(6-cyanobicyclo[3.1.0]hexan-6-yl)]benzene

A suspension of 4-(1α,5α,6β)-(6-cyanobicyclo[3.1.0]hexan-6-yl)-1-nitrobenzene (875 mg) and palladium catalyst (10% on chacoal, 87 mg) in tetrahydrofuran (19 mL) was hydrogenated at 1 atm for 3 hours at room temperature. After filtration of the catalyst, the filtrate was concentrated in vacuo to give 1-amino-4-(1α,5α,6β)-(6-cyanobicyclo[3.1.0]hexan-6-yl)benzene. To a solution of crude 1-amino-4-(1α,5α,6β)-(6-cyanobicyclo[3.1.0]hexan-6-yl)benzene thus obtained in acetone (12 mL) was added sodium hydrogencarbonate (644 mg), water (6 mL) and benzyl chloroformate (0.69 mL) at 0° C., the mixture was stirred at the same temperature for 5 minutes. After dilution of the mixture by addition of ice water, the mixture was extracted with ethyl acetate. The organic extracts were washed with brine, dried over anhydrous sodium sulfate, filtered, and then concentrated in vacuo. Flash chromatography (silica, hexane:ethyl acetate=5:2) of the residue gave 1-benzyloxycarbonylamino-4-[(1α,5α,6β)-(6-cyanobicyclo[3.1.0]hexan-6-yl)]benzene (1.27 g).

MS (EI⁺) m/z: 332 (M⁺).

HRMS (EI⁺) for C₂₁H₂₀N₂O₂ (M⁺): calcd, 332.1525; found, 332.1543.

### REFERENCE EXAMPLE 2

### 1-t-Butoxycarbonyl-3-pyrrolin-3-yl phenyl sulfone

To a suspension of N-chlorosuccinimide (781 mg) in dichloromethane (6 mL) was added benzenethiol (0.60 mL) at room temperature, the mixture was stirred at the same temperature for 30 minutes. To the resulting mixture was added a solution of 1-t-butoxycarbonyl-3-pyrroline (1.00 g) in dichloromethane (1 mL) at −60° C., the mixture was stirred at room temperature for 1 hour. The insoluble materials were filtered off, the filtrate was concentrated in vacuo. To a solution of the residue in dichloromethane (29 mL) was added m-chloroperoxybenzoic acid (3.65 g) at 0° C., the mixture was stirred at room temperature for 1 hour. To the resulting mixture was added sodium carbonate (2.19 g), the mixture was stirred at room temperature for 5 minutes. The insoluble materials were filtered off, the filtrate was diluted with ether. The filtrate was washed with 10% sodium bisulfate solution, 10% sodium carbonate solution and brine, dried over anhydrous sodium sulfate, filtered, and then concentrated in vacuo. To a solution of the residue in dichloromethane (11 mL) was added 1,8-diazabicyclo[5.4.0]undec-7-ene (0.92 mL) at −40° C., the mixture was stirred at room temperature for 5 minutes. The mixture was poured into 1 N hydrochloric acid and extracted with ether. The organic extracts were washed with water, aqueous sodium hydrogencarbonate solution and brine, dried over anhydrous sodium sulfate, filtered, and then concentrated in vacuo. Flash chromatography (silica, hexane:ethyl acetate=5:2) of the residue gave 1-t-butoxycarbonyl-3-pyrrolin-3-yl phenyl sulfone (1.16 g).

MS (EI⁺) m/z: 309 (M⁺).

HRMS (EI⁺) for C₁₅H₁₉NO₄S (M⁺): calcd, 309.1035; found, 309.1042.

### REFERENCE EXAMPLE 3

### 1-[(1α,5α,6β)-(3-t-Butoxycarbonyl-6-cyano-3-azabicyclo[3.1.0]hexan-6-yl)]-4-nitrobenzene

Step 1.

### (1α,5α,6β)-(3-t-Butoxycarbonyl-6-cyano-3-azabicyclo[3.1.0]hexan-6-yl)benzene

The title compound (1α,5α,6β)-(3-t-butoxycarbonyl-6-cyano-3-azabicyclo[3.1.0]hexan-6-yl)benzene (3.83 g) was prepared from phenylacetonitrile (1.65 mL) and 1-t-butoxycarbonyl-3-pyrrolin-3-yl phenyl sulfone (4.46 g) in the same manner as described for REFERENCE EXAMPLE 1.

MS (CI⁺) m/z: 285 (MH⁺).

HRMS (CI⁺) for C₁₇H₂₁N₂O₂ (MH⁺): calcd, 285.1603; found, 285.1616.

Step 2.

### 1-[(1α,5α,6β)-(6-Cyano-3-trifluoroacetyl-3-azabicyclo[3.1.0]hexan-6-yl)]-4-nitrobenzene

To a solution of (1α,5α,6β)-(3-butoxycarbonyl-6-cyano-3-azabicyclo[3.1.0]hexan-6-yl)benzene (853 mg) in dichloromethane (7 mL) was added trifluoroacetic acid (7 mL) at 0° C., the mixture was stirred at room temperature for 75 minutes and concentrated in vacuo. To a solution of the residue in dichloromethane (7 mL) was added triethylamine (5.01 mL) and trifluoroacetic anhydride (1.06 mL) at 0° C., the mixture was stirred at room temperature overnight and concentrated in vacuo. After dilution of the residue with ethyl acetate, the mixture was washed with 1 N hydrochloric acid, water, aqueous sodium hydrogencarbonate solution and brine, dried over anhydrous sodium sulfate, filtered, and then concentrated in vacuo. To a solution of the residue in chloroform (3 mL) was added ammonium nitrate (372 mg) and trifluoroacetic anhydride (2.19 mL), the mixture was stirred at room temperature for 2.7 hours. After addition of ice, the resulting precipitates were collected by filtration and washed with water and dichloromethane to give 1-[(1α,5α,6β)-(6-cyano-3-trifluoroacetyl-3-azabicyclo[3.1.0]hexan-6-yl)]4-nitrobenzene (546 mg).

MS (EI⁺) m/z: 325 (M⁺).

HRMS (EI⁺) for C₁₄H₁₀F₃N₃O₃ (M⁺): calcd, 325.0674; found, 325.0648.

Step 3.

### 1-[(1α,5α,6β)-(3-t-Butoxycarbonyl-6-cyano-3-azabicyclo[3.1.0]hexan-6-yl)]-4-nitrobenzene

The mixture of 1-[(1α,5α,6β)-(6-cyano-3-trifluoroacetyl-3-azabicyclo[3.1.0]hexan-6-yl))-4-nitrobenzene (9.2 mg) and a solution of ammonia in methanol (6.7 M, 0.5 mL) was stirred at room temperature for 21 hours and concentrated in vacuo. To a solution of the residue in tetrahydrofuran (0.5 mL) was added triethylamine (19.7 μL) and di-t-butyl dicarbonate (9.5 mg) at 0° C., the mixture was stirred at room temperature for 30 minutes. Preparative thin layer chromatography (silica, hexane:ethyl acetate=4:5) of the residue gave 1-[(1α,5α,6β)-(3-t-butoxycarbonyl-6-cyano-3-azabicyclo[3.1.0]hexan-6-yl)]-4-nitrobenzene (8.7 mg).

MS (EI⁺) m/z: 329 (M⁺).

HRMS (EI⁺) for C₁₇H₁₉N₃O₄(M⁺): calcd, 329.1376; found, 329.1401.

### REFERENCE EXAMPLE 4

### 1-Benzyloxycarbonylamino-4-[(1α,5α,6β)-(3-t-butoxycarbonyl-6-cyano-3-azabicyclo[3.1.0]hexan-6-yl)]benzene

The title compound 1-benzyloxycarbonylamino-4-[(1α,5α,6β)-(3-t-butoxycarbonyl-6-cyano-3-azabicyclo[3.1.0]hexan-6-yl)]benzene (127 mg) was prepared from 1-[(1α,5α,6β)-(3-t-butoxycarbonyl-6-cyano-3-azabicyclo[3.1.0]hexan-6-yl)]-4-nitrobenzene (98.8 mg) in the same manner as described for REFERENCE EXAMPLE 1.

MS (EI⁺) m/z: 433 (M⁺).

HRMS (EI⁺) for C₂₅H₂₇N₃O₄ (M⁺): calcd, 433.2002; found, 433.1989.

Antibacterial Activity

The pharmaceutically-acceptable compounds of the present invention are useful antibacterial agents having a good spectrum of activity in vitro against standard bacterial strains, which are used to screen for activity against pathogenic bacteria. Notably, the pharmaceutically-acceptable compounds of the present invention show activity against vancomycin-resistant enterococci, streptococci including penicillin-resistant S. pneumoniae, methicillin-resistant S. aureus, M. catarrhalis, and C. pneumoniae. The antibacterial spectrum and potency of a particular compound may be determined in a standard test system.

The following in vitro results were obtained based on an agar dilution method except for C. pneumoniae. The activity is presented as the minimum inhibitory concentration (MIC).

S. aureus and M. catarrhalis were tested on Mueller-Hinton agar, using an approximate inoculum of 1×10⁴ cfu/spot an incubation temperature of 35° C. for 24 hours. The MIC was defined as the lowest concentration at which no visible bacterial growth was observed.

Streptococci and enterococci were tested on Mueller-Hinton agar supplemented with 5% defibrinated horse blood, using an approximate inoculum of 1×10⁴ cfu/spot an incubation temperature of 35° C. in an atmosphere of 5% CO₂ for 24 hours. The MIC was defined as the lowest concentration at which no visible bacterial growth was observed.

C. pneumoniae was tested using minimum essential medium supplemented with 10% heat-inactivated fetal bovine serum, 2 mM L-glutamine, 1 mg/ml cycloheximide and non essential amino acid. HeLa 229 cells were inoculated with 10⁴ inclusion-forming units of C. pneumoniae strain per mL. Infected cells were incubated with test compounds in complete medium at 35° C. in an atmosphere of 5% CO₂ for 72 hours. Cells monolayers were fixed in methanol, stained for chlamydial inclusions with a fluorescein-conjugated anti-Chlamydia monoclonal antibody, and were observed with fluorescence microscope. The MIC was defined as the lowest concentration at which no inclusion was observed.

| | MIC (μg/ml) | | | | |
|---|---|---|---|---|---|
| | example | example | | | |
| Strains | 1 | 8 | example 9 | example 11 | Linezolid |
| Staphylococcus | | | | | |
| aureus | | | | | |
| Smith | 0.25 | 1 | 0.5 | 0.25 | 1 |
| CR | 2 | 2 | 1 | 1 | 16 |
| MR | 0.25 | 1 | 0.5 | 0.5 | 1 |
| Streptococcus | | | | | |
| pneumoniae | | | | | |
| IID553 | 0.5 | 0.5 | 0.5 | 0.25 | 2 |
| PRQR | 0.25 | 0.5 | 0.5 | 0.25 | 1 |
| Streptococcus | | | | | |
| pyogenes | | | | | |
| IID692 | 0.5 | 0.5 | 0.5 | 0.125 | 1 |
| Enterococcus | | | | | |
| faecium | | | | | |
| VRQR | 0.25 | 0.5 | 0.5 | 0.25 | 2 |
| Moraxella | | | | | |
| catarrhalis | | | | | |
| ATCC25238 | 0.5 | 2 | 2 | 1 | 4 |

CR = chloramphenicol resistant
MR = methicillin resistant
PRQR = penicillin resistant, quinolone resistant
VRQR = vancomycin resistant, quinolone resistant
NT = not tested

The invention described herein is exemplified by the following non-limiting examples. The compound data is designated in accordance to General Guidelines for Manuscript Preparation, J. Org. Chem Vol. 66, pg. 19A, Issue 1, 2001.

1. The present invention relates to compounds of formula I: 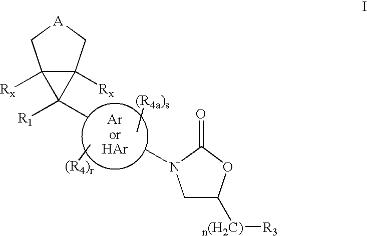
its enantiomer, diastereomer, or pharmaceutically acceptable salt, hydrate or prodrug thereof wherein:
R₁ represents
i) hydrogen,
ii) NR₅R₆,
iii) CR₇R₈R₉, C(R)₂OR₁₄, CH₂NHR₁₄,
iv) C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄,
v) (C₁₋₆alkyl)CN, CN, CH═C(R)₂, (CH₂)ₚOH, C(═O)CHR₁₃, C(═NR₁₃)R₁₃, NR₁₀C(═X₁)R₁₃;
vi) C₅₋₁₀ heterocycle optionally substituted with 1-3 groups of R_{7,} which may be attached through either a carbon or a heteroatom;
A represents NR, O, or S(O)p; 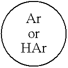
represents aryl or heteroaryl, provided that in the case of a heteroaryl, a cyclopropyl is not attached to a nitrogen atom on the ring;
Rₓ represents hydrogen or C₁₋₆ alkyl;
R₃ represent
i) NR₁₃(C═X₂)R₁₂,
ii) NR₁₃(C═X₁)R₁₂,
iii) NR₁₃SO₂R₁₄,
iv) N(R₁₃)heteroaryl,
v) NR₁₃(CHR₁₃)₀₋₄aryl,
vi) NR₁₃(CHR₁₃)₀₋₄heteroaryl,
vii) S(CHR₁₃)₀₋₄aryl,
viii) S(CHR₁₃)₀₋₄heteroaryl,
ix) O(CHR₁₃)₀₋₄aryl,
x) O(CHR₁₃)₀₋₄heteroaryl,
xi) NOH(C═X₁)R₁₂,
xii) —OC═N(OCOaryl)C₁₋₆ alkyl
xiii) —OC═N(OH)C₁₋₆ alkyl
xiv) C₅₋₁₀ heteroaryl which may be attached through either a carbon or a heteroatom; said aryl and heteroaryl optionally substituted with 1-3 groups of R_{7,}
R₄, and R_{4a,} independently represent
i) hydrogen,
ii) halogen,
iii) C₁₋₆ alkoxy, or
iv) C₁₋₆ alkyl
r and s independently are 1-3, with the provision that when (R₄ₐ)ₛ and (R₄)ᵣ are attached to an Ar or HAr ring the sum of r and s is less than or equal to 4;
R₅ and R₆ independently represent
i) hydrogen,
ii) C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;
iii) C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
iv) C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl;
v) arylsulfonyl optionally substituted with 1-3 of halogen, C₁₋₆ alkoxy, OH or C₁₋₆ alkyl;
vi) C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl;
vii) aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl
viii) five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
ix) C₃₋₆ cycloalkylcarbonyl optionally substituted with 1-3 groups of halogen, OH, C₁₋₆ alkoxy or CN;
x) benzoyl optionally substituted with 1-3 groups of halogen, OH, C₁₋₆ alkoxy, C₁₋₆ alkyl, CF₃, C₁₋₆ alkanoyl, amino or C₁₋₆ acylamino;
xi) pyrrolylcarbonyl optionally substituted with 1-3 of C₁₋₆ alkyl;
xii) C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or
R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
i) hydrogen, halogen, CN, CO₂R, CON(R)₂, CHO, CH₂NHAc, C(═NOR), OH, C₁₋₆ alkoxy, C₁₋₆ alkyl, alkenyl, hydroxy C₁₋₆ alkyl, (CH₂)₁₋₃NHC(O)C₁₋₆ alkyl, (CH₂)₁₋₃N(C₁₋₆ alkyl)₂
ii) (CH₂)ₙamino, (CH₂)ₙC₁₋₆ alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
i) H, CN,
ii) C₁₋₆ alkyl optionally substituted with 1-3 halogen, CN, OH, C₁₋₆ alkoxy, C₁₋₆ acyloxy, or amino,
iii) phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
X₂ represents O, S, NH or NSO₂R₁₄;
R₁₀ represents hydrogen, C₁₋₆ alkyl or CO₂R₁₅;
R₁₂ represents hydrogen, C₁₋₆ alkyl, NH₂, OR, CHF₂, CHCl₂, CR₂Cl, (CH₂)ₙSR, (CH₂)ₙCN, (CH₂)ₙSO₂R, (CH₂)ₙS(O)R, C₁₋₆ alkylamino, C₅₋₁₀ heteroaryl or C₁₋₆ dialkylamino, where said alkyl may be substituted with 1-3 groups of halo, CN, OH or C₁₋₆ alkoxy, said heteroaryl optionally substituted with 1-3 groups of R₇;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, —OCOaryl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen or C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
p represents 0-2 and
m, n, and q represents 0-1.

2. A compound according to claim 1 wherein R₁ represents H, NR₅R₆, CN, OH, C(R)₂OR₁₄, NHC(═X₁)N(R₁₃)₂, C(═NOH)N(R₁₃)₂, NR₁₀C(═X₁)R₁₃ or CR₇R₈R₉.

3. A compound according to claim 1 wherein 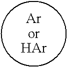
is phenyl, pyridine, pyrimidine, or piperidine.

4. A compound according to claim 3 wherein R₁ is NR₅R₆, or CN and R3 is NR₁₀C(═X₁)R₁₃, NR(C═X₁)R₁₂, C₅₋₁₀ heteroaryl, NH(CH₂)₀₋₄aryl, NH(CH₂)₀₋₄heteroaryl, said aryl and heteroaryl optionally substituted with 1-3 groups of Ra.

5. A compound according to claim 3 wherein R₃ is a C₅₋₁₀ heteroaryl represented by 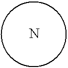
which represents an optionally substituted aromatic heterocyclic group containing 1 to 4 nitrogen atoms and at least one double bond, and which is connected through a bond on any nitrogen.

6. A compound according to claim 1 wherein the structural formula is II: 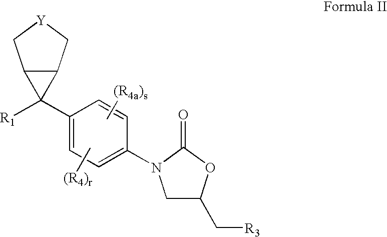
wherein R₁, R₄, R₄ₐ, Y and R₃ are as described above.

7. A compound which is:
N-[5(S)-3-[4-[(1α,5α,6β)-(6-cyanobicyclo[3.1.0]hexan-6-yl)]phenyl]-2-oxooxazolidin-5-ylmethyl]acetamide,
1-[5(R)-3-[4-[(1α,5α,6β)-(6-cyanobicyclo[3.1.0]hexan-6-yl)]phenyl]-2-oxooxazolidin-5-ylmethyl]-1,2,3-triazole,
N-[5(S)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-azabicyclo[3.1.0]hexan-6-yl)]phenyl]-2-oxooxazolidin-5-ylmethyl]acetamide,
1-[5(R)-3-[4-[(1α,5α,6β)-(3-t-butoxycarbonyl-6-cyano-3-azabicyclo[3.1.0]hexan-6-yl)]phenyl]-2-oxooxazolidin-5-ylmethyl]-1,2,3-triazole,
1-[5(R)-3-[4-[(1α,5α,6β)-(6-cyano-3-azabicyclo[3.1.0]hexan-6-yl)]phenyl]-2-oxooxazolidin-5-ylmethyl]-1,2,3-triazole,
N-[5(S)-3-[4-[(1α,5α,6β)-(3-acetoxyacetyl-6-cyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-hydroxyacetyl-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-methanesulfonyl-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-methyl-3-azabicyclo[3.1.0]hexan-6-yl)]phenyl]-2-oxooxazolidin-5-ylmethyl]acetamide,
N-[5(S)-3-[4-[(1α,5α,6β)-(3,6-dicyano-3-azabicyclo[3.1.0]hexan-6-yl)]phenyl]-2-oxooxazolidin-5-ylmethyl]acetamide,
N-[5(S)-3-[4-[(1α,5α,6β)-(6-cyano-3-cyanomethyl-3-azabicyclo[3.1.0]hexan-6-yl)]phenyl]-2-oxooxazolidin-5-ylmethyl]acetamide,
5(R)-3-[4-[(1α,5α,6β)-(3-t-butoxycarbonyl-6-cyano-3-azabicyclo[3.1.0]hexan-6-yl)]phenyl]-5-[(isoxazol-3-yl)oxy]methyloxazolidin-2-one,
5(R)-3-[4-[(1α,5α,6β)-(6-cyano-3-azabicyclo[3.1.0]hexan-6-yl)]phenyl]-5-[(isoxazol-3-yl)oxy]methyloxazolidin-2-one,
5(R)-3-[4-[(1α,5α,6β)-(3-t-butoxycarbonyl-6-cyano-3-azabicyclo[3.1.0]hexan-6-yl)]phenyl]-5-[N-(t-butoxycarbonyl)-N-(isoxazol-3-yl)]aminomethyloxazolidin-2-one,
5(R)-3-[4-[(1α,5α,6β)-(6-cyano-3-azabicyclo[3.1.0]hexan-6-yl)]phenyl]-5-[N-(isoxazol-3-yl)]aminomethyloxazolidin-2-one,
N-[5(S)-3-[4-[(1α,5α,6β)-[6-cyano-3-(5-cyanopyridin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(pyridin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[3-acetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(pyrimidin-2-yl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(4-pyridylmethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyano-1-iminoethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-methoxycarbonyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyano-S-methylthioiminomethyl)-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(N-cyanocarboxamidyl)-3-azabicyclo[3.1.0]hexan-6-yl]]phenyl]-2-oxooxazolidin-5-ylmethyl]acetamide,
N-[5(S)-3-[4-[(1α,5α,6β)-[3-(N,N′-t-butoxycarbonylcarboxamidyl)-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-carboxamidyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-(N-t-Butoxycarbonylamino)acetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[3-aminoacetyl-6-cyano-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-methanesulfonylacetyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(dibenzylphosphoryloxy)acetyl-3-azabicyclo[3.1.0]hexan-6-yl]]phenyl]-2-oxooxazolidin-5-ylmethyl]acetamide,
N-[5(S)-3-[4-[(1α,5α,6β)-[6-cyano-3-(phosphoryloxy)acetyl-3-azabicyclo[3.1.0]hexan-6-yl]]phenyl]-2-oxooxazolidin-5-ylmethyl]acetamide,
or their enantiomer, diastereomer, or pharmaceutically acceptable salt, hydrate or prodrug thereof wherein.

8. A pharmaceutical composition comprised of a compound in accordance with claim 1 in combination with a pharmaceutically acceptable carrier and optionally a in combination with a vitamin selected from the group consisting vitamin B2, vitamin B6, vitamin B12 and folic acid.

9. A method of treating or preventing a bacterial infection in a mammalian patient in need thereof, comprising administering to said patient an effective amount of a compound of claim 1.

10. A method of treating or preventing bacterial infection or an oxazolidinone-associated side effect by administering an effective amount of a compound of formula I of claim 1 and an effective amount of one or more of a vitamin selected from the group consisting of vitamin B2, vitamin B6, vitamin B 12 and folic acid to a patient in need thereof.

11. A method according to claim 1 for treating or preventing oxazolidinone-associated normocyctic anemia, peripheral sensory neuropathy, sideroblastic anemia, peripheral sensory neuropathy, optic neuropathy, seizures, thrombocytopenia, cheilosis, hypo-regenerative anemia, megaloblastic anemia and seborrheic dermatitis by administering an effective amount of vitamin B2 to a patient in need thereof.
